(19) United States
(12) Patent Application Publication
(10) Pub. No.: US 2003/0138425 A1
(43) Pub. Date: Jul. 24, 2003

(54) Antibodies that bind to cancer-associated antigen cytokeratin 8 and methods of use thereof

(76) Inventor: Jennie Powell Mather, Millbrae, CA (US)

Correspondence Address: Jie Zhou, Morrison & Foerster LLP, 755 Page Mill Road, Palo Alto, CA, 94304-1018, US

(21) Appl. No.: 10/253,118

(22) Filed: Sep. 23, 2002

Related U.S. Application Data
(60) Provisional application No. 60/323,844, filed on Sep. 21, 2001.
(60) Provisional application No. 60/408,253, filed on Sep. 4, 2002.

(51) Int. Cl. G01N 33/574; A61K 39/395; C07H 21/04; C12P 21/02; C07K 16/40; C12N 5/06

(52) U.S. Cl. 424/146.1; 530/388.26; 435/69.1; 435/334; 435/320.1; 435/7.23; 536/23.53

(57) ABSTRACT

Provided herein is disclosure about the development and characterization of an antibody (mhoe-4) which binds to antigen cytokeratin 8, which is present on a variety of human cancers such as ovarian, breast, lung, prostate, colon, kidney, thyroid, bone, upper digestive tract, and pancreatic cancers. Methods of diagnosing and treating various cancers by using antibodies such as mhoe-4 directed against this antigen are also disclosed.

### CROSS-REFERENCE TO RELATED APPLICATIONS

This application claims the benefit of U.S. provisional application serial No. 60/323,844, filed Sep. 21, 2001, and U.S. provisional application No. 60/408,253, filed Sep. 4, 2002, which are incorporated in their entirety by reference.

### TECHNICAL FIELD

This invention is in the fields of cancer biology and immunotherapy. More specifically, it concerns the discovery of antibodies that bind to cytokeratin 8, which is present in a variety of human cancers, and methods of diagnosing and/or treating such cancers.

### BACKGROUND OF THE INVENTION

Immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6^{th} Edition (2001) Chapt. 20 pp. 495-508. These antibodies can have inherent therapeutic biological activity both by direct inhibition of tumor cell growth or survival and by their ability to recruit the natural cell killing activity of the body's immune system. These agents can be administered alone or in conjunction with radiation or chemotherapeutic agents. Rituxan® and Herceptin®, approved for treatment of lymphoma and breast cancer, respectively, are two examples of such therapeutics. Alternatively, antibodies can be used to make antibody conjugates where the antibody is linked to a toxic agent and directs that agent to the tumor by specifically binding to the tumor. Mylotarg® is an example of an approved antibody conjugate used for the treatment of leukemia. Monoclonal antibodies that bind to cancer cells and have potential uses for diagnosis and therapy have been disclosed in publications. See, for example, the following patent applications which disclose, inter alia, some molecular weights of target proteins: U.S. Pat. No. 6,054,561 (200 KD c-erbB-2 (Her2), and other unknown antigens 40-200 KD in size) and U.S. Pat. No. 5,656,444 (50 KD and 55 KD, oncofetal protein). Example of antibodies in clinical trials and/or approved for treatment of solid tumors include: Herceptin (antigen: 180 kD, HER2/neu), Panorex (antigen: 40-50 kD, Ep-CAM), HMFG1 (antigen>200 kD, HMW Mucin), and C225 (antigens:150 kD and 170 kD, EGF receptor).

Another type of immunotherapy is active immunotherapy, or vaccination, wherein the antigen present on a specific cancer(s) or a DNA construct that directs the expression of the antigen, which then evokes the immune response in the patient, i.e., to induce the patient to actively produce antibodies against their own cancer. Active immunization has not been used as often as passive immunotherapy or immunotoxins.

An ideal diagnostic and/or therapeutic antibody would be specific for an antigen present on a large number of cancers, but absent or present only at low levels on any adult tissue. An antibody would ideally have biological activity against cancer cells and be able to recruit the immune system's response to foreign antigens. An antibody could be administered as a therapeutic alone or in combination with current treatments or used to prepare immunoconjugates linked to toxic agents. An antibody with the same specificity but without biological activity when administered alone could also be useful in that an antibody could be used to prepare an immunoconjugate with a radio-isotope, a toxin, or a chemotherapeutic agent or liposome containing a chemotherapeutic agent, with the conjugated form being biologically active by virtue of the antibody directing the toxin to the antigen-containing cells.

Antibodies to cytokeratin 8, a member of the intermediate filament group of cytoskeletal proteins, have been reported. See, for example, U.S. Pat. Nos. 5,180,814; 5,338,661; 5,399,482; 5,474,755; 5,489,590; 5,660,994; 5,780,032; 6,168,779; 6,190,870; and 6,200,765. The expression of cytokeratin 8 (CK8) has been reported to be present in certain types of cancer, such as breast cancer (Lehr et al., 2000, Am. J. Pathol. 114(2):190-96), colon cancer (Nishibori et al., 1996, Anticancer Res. 56(12):2752-57), lung cancer (ten Velde et al., 1990, Eur J. Cancer 26(11-12):1142-45), ovarian cancer (Van Niekirk et al., 1993, Am. J. Pathol. 142(1):157-77, and Yanagibashi et al., 1997, Br. J. Cancer 76(7):829-35), pancreatic cancer (Luttges et al., 1998, Histopathology 32(5):444-48), prostate cancer (Silen et al., 1994, Prostate 24(6):326-32), and renal cancer (Ishii et al., 1989, Cancer Res. 49(19):5392-99, Oosterwijk et al., 1990, J. Immunohistochem. Cytochem. 38(3):385-92). Antibodies that are specific for CK8 on cancerous cells would be useful for treatment and diagnosis.

All patents, patent applications, and publications cited herein are hereby incorporated by reference in their entirety.

### BRIEF SUMMARY OF THE INVENTION

The invention disclosed herein concerns antibodies to an antigen, cytokeratin 8 (CK8), which is present in a variety of human cancers. Accordingly, in one aspect, the invention is an antibody or a polypeptide (which may or may not be an antibody) that binds preferentially to the antigen, hereinafter known as “Ag-hoe4” or cytokeratin 8 (CK8), which is approximately 51 kDa +/−10% on a 4-20% Tris-glycine SDS-PAGE (i.e., denaturing gradient) gel.

In another aspect, the invention is an antibody or a polypeptide (which may or may not be an antibody) that binds preferentially to CK8 but does not bind preferentially to cytokeratin 18 (CK18).

In another aspect, the invention is an antibody or a polypeptide (which may or may not be an antibody) that preferentially binds to one or more peptides selected from the group consisting of FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7), or to polypeptides comprising one or more of these sequences. These peptide sequences are contained in CK8. In one embodiment, the antibody binds preferentially to one of the seven peptides. In some embodiments, the antibody binds preferentially to the peptide FLEQQNKMLETK (SEQ ID NO:1). In another embodiment, the antibody the antibody binds preferentially to two of the seven peptides. In yet another embodiment, the antibody binds preferentially to three of the seven peptides. In yet another embodiment, the antibody binds preferentially to four of the seven peptides. In yet another embodiment, the antibody binds preferentially to five of the seven peptides. In yet another embodiment, the antibody binds preferentially to six of the seven peptides. In yet another embodiment, the antibody binds preferentially to all of the seven peptides.

In another aspect, the invention is an antibody mhoe-4 that is produced by a host cell with a deposit number of ATCC No. PTA-3159 or progeny thereof. Antibody mhoe-4 binds to cytokeratin 8 (CK8). Antibody mhoe-4 preferentially binds to CK8 but does not bind preferentially to CK18. CK8 peptide sequences that mhoe-4 binds include FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7).

In another aspect, the invention is an antibody or a polypeptide (which may or may not be an antibody) that binds preferentially to the epitope that mhoe-4 preferentially binds.

In another aspect, the invention is an antibody comprising a fragment or region of an antibody mhoe-4. In one embodiment, the fragment is a light chain of the antibody mhoe-4. In another embodiment, the fragment is a heavy chain of the antibody mhoe-4. In yet another embodiment, the fragment contains one or more variable regions from a light chain and/or a heavy chain of the antibody mhoe-4. In yet another embodiment, the fragment contains one or more complementarity determining regions (CDRs) from a light chain and/or a heavy chain of the antibody mhoe-4.

In another aspect, the invention provides polypeptides (which may or may not be antibodies) comprising any of the following: a) one or more CDRs; b) three CDRs from the light chain; c) three CDRs from the heavy chain; d) three CDRs from the light chain and three CDRs from the heavy chain; e) the light chain variable region; f) the heavy chain variable region of the antibody mhoe-4.

In another aspect, the invention is a humanized antibody comprising one or more CDRs of the antibody mhoe-4. In another aspect, the invention provides a humanized antibody that binds to the same epitope(s) as antibody mhoe-4. Generally, a humanized antibody of the invention comprises one or more (one, two, three, four, five, six) CDRs which are the same and/or derived from the CDR(s) of antibody mhoe-4. In other aspect, the invention provides a human antibody which binds to the same epitope(s) as antibody mhoe-4.

In another aspect, the invention is a host cell line (ATCC No. PTA-3159) or progeny thereof that produces monoclonal antibody mhoe-4.

In another aspect, the invention is an isolated polynucleotide that encodes for antibody mhoe-4 that is produced by a host cell with a deposit number of ATCC No. PTA-3159 or progeny thereof. In another aspect, the invention provides polynucleotides encoding any of the antibodies (including antibody fragments) as well as any other polypeptides described herein.

In another aspect, the invention is a complex of Ag-hoe4 (cytokeratin 8) bound by antibody mhoe-4. In some embodiments, the CK8 is present breast, colon, lung, ovarian, pancreatic, prostate, renal, or thyroid cancer. In one embodiment, antibody mhoe-4 is linked to a therapeutic agent (such as a toxin). In another aspect, the invention provides a complex of any of the antibody or polypeptides described herein and CK8.

In another aspect, the invention is a pharmaceutical composition comprising any of the polypeptides (including any of the antibodies such as antibody mhoe-4) or polynucleotides described herein, such as pharmaceutical compositions comprising the antibody mhoe-4, the antibody mhoe-4 linked to a therapeutic agent, an antibody comprising a fragment of the antibody mhoe-4, a humanized antibody of the antibody mhoe-4, or a human antibody with one or more properties of the antibody mhoe-4, and a pharmaceutically acceptable excipient.

In another aspect, the invention is a method of generating a monoclonal antibody having greater affinity to cancerous cells than non-cancerous cells by: (a) immunizing a host mammal with intact human fetal ovarian epithelial (HOE) cells; (b) obtaining lymphocytes from the mammal; (c) fusing lymphocytes with a myeloma cell line to produce a hybridoma; (d) culturing the hybridoma under conditions sufficient to produce monoclonal antibodies; (e) selecting antibodies that preferentially bind to HOE cells; and (f) selecting from the antibodies obtained from step (e) an antibody that binds to cancerous cells with greater affinity than non-cancerous cells.

In another aspect, the invention is a method of generating antibody mhoe-4 comprising culturing a host cell (ATCC No. PTA-3159) or progeny thereof under conditions that allow production of antibody mhoe-4, and purifying the antibody mhoe-4.

In another aspect, the invention provides methods of generating any of the antibodies (or polypeptides) described herein by expressing one or more polynucleotides encoding the antibody (which may be separately expressed as a single light or heavy chain) in a suitable cell, generally followed by isolating the antibody or polypeptides of interest.

In another aspect, the invention is a method of diagnosing (e.g., detecting or identifying presence or absence) thyroid cancer in an individual by detecting Ag-hoe4 (cytokeratin 8) from thyroid cells from the individual. For these embodiments, any antibody (or moiety which binds CK8) may be used. In one embodiment, cytokeratin 8 (CK8) from cells are detected by the antibody mhoe-4 or any CK8 binding moiety (polypeptides, including, but not limited to, various antibodies and antibody derivatives) described herein. The term “detection” as used herein include qualitative and/or quantitative detection (measuring levels) with or without reference to a control.

In another aspect, the invention is a method of diagnosing cancer in an individual by detecting cytokeratin 8 from cells from the individual using the antibody mhoe-4 or any CK8 binding moiety (polypeptides, including, but not limited to, various antibodies and antibody derivatives) described herein. In some embodiments, the cancer is breast, colon, lung, ovarian, pancreatic, prostate, renal, or thyroid. In some embodiments, the method is detecting the level of cytokeratin 8 from cells. The presence of cytokeratin 8 is detected by detecting a complex between cytokeratin 8 and a CK8 binding moiety.

In another aspect, the invention is a method of treating cancer by administering an effective amount of a composition comprising the antibody mhoe-4, or any of the antibodies (including polypeptides) or polynucleotides embodiments described herein, including but not limited to the antibody mhoe-4 associated with a therapeutic agent, an antibody comprising a fragment of the antibody mhoe-4, or a humanized antibody (generally, but not necessarily, comprising one or more CDRs of the antibody mhoe-4), sufficient to reduce growth of cancerous cells. In some embodiments, the cancer is breast, colon, lung, ovarian, pancreatic, prostate, renal, or thyroid.

In another aspect, the invention is a method of inhibiting growth and/or proliferation of cancerous cells in an individual by administering to the individual an effective amount of a composition comprising the antibody mhoe-4, or any of the antibodies (including polypeptides) or polynucleotides embodiments described herein, including but not limited to the antibody mhoe-4 associated with a therapeutic agent, an antibody comprising a fragment of the antibody mhoe-4, or a humanized antibody (generally, but not necessarily, comprising one or more CDRs of the antibody mhoe-4), sufficient to reduce growth of cancerous cells. In some embodiments, the cancer is breast, colon, lung, ovarian, pancreatic, prostate, renal, or thyroid.

In another aspect, the invention is a method of delaying development of metastasis in an individual with cancer by administering an effective amount of a composition comprising the antibody mhoe-4, or any of the antibodies (including polypeptides) or polynucleotides embodiments described herein, including but not limited to the antibody mhoe-4 associated with a therapeutic agent, an antibody comprising a fragment of the antibody mhoe-4, or a humanized antibody (generally, but not necessarily, comprising one or more CDRs of the antibody mhoe-4), sufficient to reduce growth of cancerous cells. In some embodiments, the cancer is breast, colon, lung, ovarian, pancreatic, prostate, renal, or thyroid.

In another aspect, the invention is a method of delivering a therapeutic agent (such as a toxin, or a radioactive molecule) to cancerous cells in an individual by administering to the individual an effective amount of a CK8 binding antibody or any CK8 binding moiety (polypeptides, including but not limited to antibodies or antibody derivatives) described herein that are linked to a therapeutic agent (such as a toxin or a radioactive molecule). In one embodiment, the CK8 binding antibody is mhoe-4. In another embodiment, the CK8 binding antibody is an antibody that binds to one or more of the peptides FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7). In another embodiment, the therapeutic agent (such as a toxin or a radioactive molecule) is delivered into the cancerous cells (is internalized). In another embodiment the therapeutic agent is delivered into prostate cancer cells. Accordingly, the invention provides methods of inhibiting growth and/or proliferation of prostate cancer cells such that the therapeutic agent is delivered into prostate cancer cells. In another embodiment, the therapeutic agent is saporin.

In another aspects, the invention provides kits comprising any one or more of the compositions described herein. These kits, generally in suitable packaging and provided with appropriate instructions, and useful for any of the methods described herein.

### BRIEF DESCRIPTION OF THE DRAWING(S)

FIG. 1 is a photograph that shows mhoe-4 staining of three primary ovarian carcinomas and one metastatic nodule (met, lower right). Dark circles of staining indicate that the staining is cell surface in nature.

FIG. 2 is a photograph that shows mhoe-4 staining of a metastatic breast carcinoma.

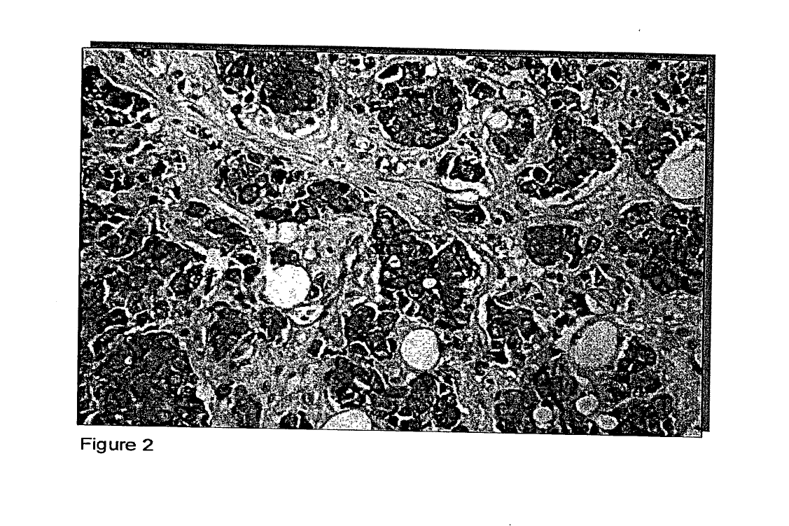

FIG. 3 is a photograph that shows mhoe-4 staining of a metastatic prostate carcinoma (A, B), a primary prostate cancer (C) and adjacent non-cancerous prostate (D).

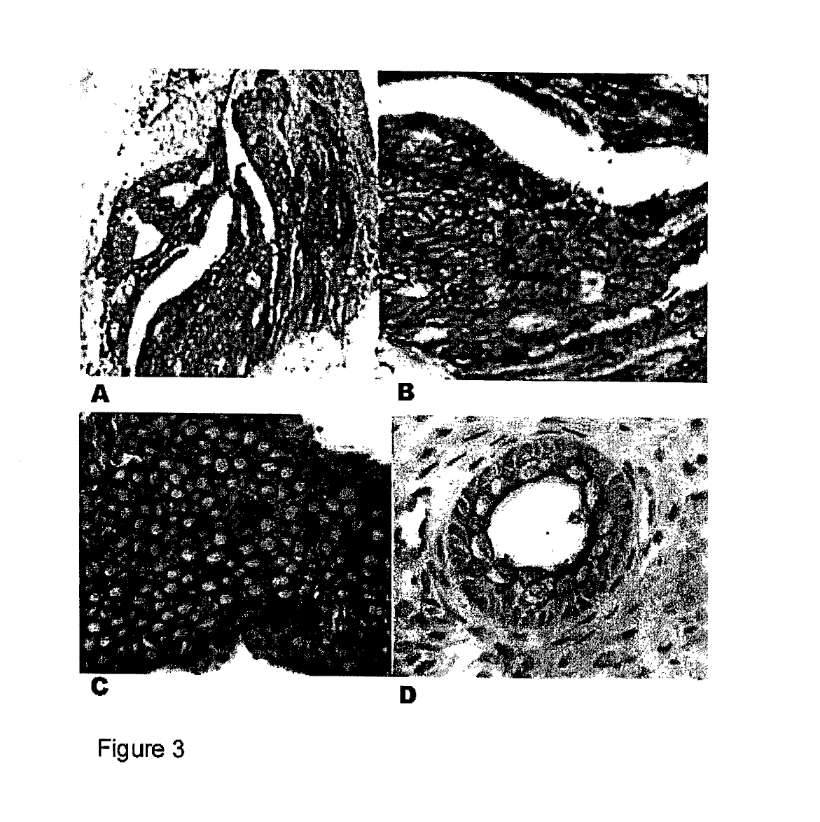

FIG. 4 is a graph that shows the effect of mhoe-4 and Mab-ZAP (an anti-IgG conjugated to saporin) on the growth human prostate tumor cells LNCaP.

### DETAILED DESCRIPTION OF THE INVENTION

The invention disclosed herein provides antibodies and polypeptides which bind to an antigen, cytokeratin 8 (CK8) and methods of making and using these antibodies and polypeptides which bind to cytokeratin 8. Cytokeratin 8 has been shown to be present and its expression is increased in a variety of human cancers. An antibody mhoe-4, which binds preferentially to cytokeratin 8 but does not bind preferentially to cytokeratin 18, has been found to suppress tumor growth in vitro and in an in vivo model and has displayed an ability to internalize a therapeutic agent in prostate cancer cells.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, humanized antibodies, chimeric antibodies, and any other modified configuration of the immunoglobulin molecule that comprises an antigen recognition site of the required specificity.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and fill-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody, which are also termed one or more CDRs “derived-from”-one or more CDRs from mhoe-4.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a cytokeratin 8 (CK8) epitope is an antibody that binds this CK8 epitope with greater affinity, avidity, more readily, and/or with greater duration than it binds to other CK8 epitopes or non-CK8 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the terms “mhoe-4”, “antibody mhoe-4” and “monoclonal antibody mhoe-4” are used interchangeably to refer to immunoglobulin produced by a host cell with a deposit number of ATCC No. PTA-3159 or progeny thereof. The generation and characterization of mhoe-4 is described in Examples. Different biological functions are associated with mhoe-4, including, but not limited to, ability to bind to cytokeratin 8 (CK8); ability to preferentially bind to CK8 but not to preferentially bind to cytokeratin 18 (CK18) (i.e., as compared to binding to CK18); ability to bind to one or more peptides FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7); ability to inhibit growth of cancerous cells expressing CK8, such as ovarian or prostate cancer cells; ability to delay development of metastasis in an individual with cancerous cells expressing CK8; ability to deliver a therapeutic agent, such as a toxin or a radioactive compound to cancerous cells expressing CK8; ability to deliver a therapeutic agent into cancerous cells expressing CK8, such as prostate cancer cells. As discussed herein, polypeptides (including antibodies) of the invention may have any one or more of these characteristics.

A “mhoe-4 equivalent antibody” or “mhoe-4 equivalent polypeptide” refers to an antibody or a polypeptide having one or more biological functions associated with mhoe-4.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

A “variable region” of an antibody refers to the variable region of the antibody light chain or the variable region of the antibody heavy chain, either alone or in combination.

As used herein, “substantially pure” refers to material which is at least 50% pure (i.e., free from contaminants), more preferably at least 90% pure, more preferably at least 95% pure, more preferably at least 98% pure, more preferably at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

An “effective amount” of drug, compound, or pharmaceutical composition is an amount sufficient to effect beneficial or desired results including clinical results such as shrinking the size of the tumor, retardation of cancerous cell growth, decreasing one or more symptoms resulting from the disease, increasing the quality of life of those suffering from the disease, decreasing the dose of other medications required to treat the disease, delaying the progression of the disease, and/or prolonging survival of patients. An effective amount can be administered in one or more administrations. For purposes of this invention, an effective amount of drug, compound, or pharmaceutical composition is an amount sufficient to reduce the proliferation of (or destroy) neoplastic cells and/or to reduce and/or delay the development, or growth, of metastases of neoplastic cells, either directly or indirectly. As is understood in the cancer clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) neoplastic cells, reducing metastasis of neoplastic cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of patients.

As used herein, “delaying development of metastasis” means to defer, hinder, slow, retard, stabilize, and/or postpone development of metastasis. This delay can be of varying lengths of time, depending on the history of the cancer and/or individual being treated. As is evident to one skilled in the art, a sufficient or significant delay can, in effect, encompass prevention, in that the individual does not develop the metastasis.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

An “individual” is a vertebrate, preferably a mammal, more preferably a human. Mammals include, but are not limited to, farm animals, sport animals, pets, primates, mice and rats.

“Toxin” or “cytotoxin” refers to any substance which effects an adverse response within a cell. For example, a toxin directed to a cancerous cell would have an adverse, sometimes deleterious effect, on the cancerous cell.

As used herein, “agent” refers to a biological, pharmaceutical, or chemical compound. Non-limiting examples include simple or complex organic or inorganic molecule, a peptide, a protein, an oligonucleotide, an antibody, an antibody derivative, or antibody fragment. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like.

As used herein, a “therapeutic agent” means any agent useful for therapy (here, generally in the cancer context) including anti-tumor drugs, toxins or cytotoxins, cytotoxin agents, and radioactive agents.

“Active immune response” refers to the development of, and on-going production of, antibodies in vivo directed against an antigen, in response to the administration of the antigen, or DNA vectors coding for that antigen, to the host mammal by intravenous, intramuscular, subcutaneous, or other mode of administration with or without an adjuvant. Active immune response can also include other aspects of the immune response, such as a cellular immune response.

Compositions and Methods of Making the Compositions

This invention encompasses compositions, including pharmaceutical compositions, comprising antibodies, polypeptides and proteins that bind to cytokeratin 8 (CK8), and polynucleotides comprising sequences encoding antibodies, polypeptides and proteins that bind to cytokeratin 8 (CK8). As used herein, compositions comprise one or more antibodies, polypeptides and proteins that bind to CK8, and one or more polynucleotides comprising sequences encoding one or more antibodies, polypeptides and proteins that bind to cytokeratin 8 (CK8). These compositions may further comprise suitable excipients, such as pharmaceutically acceptable excipients including buffers, which are well known in the art.

The CK8 binding antibodies, polypeptides and proteins of this invention are further identified and characterized by any (one or more) of the following criteria: (a) ability to preferentially bind to cytokeratin 8 (CK8), but not to preferentially bind to cytokeratin 18 (CK18); (b) the ability to preferentially bind to one or more of peptides FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7); (c) ability to inhibit proliferation and/or growth of cancerous cells expressing CK8, such as prostate or ovarian cancer cells; (d) ability to delay development of metastasis in an individual with cancer expressing CK8; (e) ability to deliver a therapeutic agent, such as a toxin, or a radioactive compound, to cancerous cells expressing CK8; (f) ability to deliver a therapeutic agent into cancerous cells expressing CK8, such as prostate cancer cells.

In some embodiments, the antibodies, polypeptides and proteins of the invention that bind to CK8 are antibodies, polypeptides and proteins that preferentially bind to one or more peptides selected from the group consisting of a) FLEQQNKMLETK (SEQ ID NO:1), b) QEKEQIKTLNNK (SEQ ID NO:2), c) YQELMNVKLALD (SEQ ID NO:3), d) NMQGLVEDFKNK (SEQ ID NO:4), e) PRAFSSRSYTSG (SEQ ID NO:5), f) SSAYGGLTSPGL (SEQ ID NO:6), and g) EDIANRSRAEAE (SEQ ID NO:7). See Example 7. In some embodiments, the antibodies, polypeptides and proteins of the invention that bind to CK8 are antibodies, polypeptides and proteins that bind preferentially to peptide a). In some embodiments, the antibodies, polypeptides and proteins of the invention that bind to CK8 are antibodies, polypeptides and proteins that bind preferentially to peptide b). In some embodiments, the antibodies, polypeptides and proteins of the invention that bind to CK8 are antibodies, polypeptides and proteins that bind preferentially to peptide c). In some embodiments, the antibodies, polypeptides and proteins of the invention that bind to CK8 are antibodies, polypeptides and proteins that bind preferentially to peptide d). In some embodiments, the antibodies, polypeptides and proteins of the invention that bind to CK8 are antibodies, polypeptides and proteins that bind preferentially to peptide e). In some embodiments, the antibodies, polypeptides and proteins of the invention that bind to CK8 are antibodies, polypeptides and proteins that bind preferentially to peptide f). In some embodiments, the antibodies, polypeptides and proteins of the invention that bind to CK8 are antibodies, polypeptides and proteins that bind preferentially to peptide g). In some embodiments, the antibodies, polypeptides and proteins of the invention that bind to CK8 are antibodies, polypeptides and proteins that bind preferentially to peptide a) and b). In some embodiments, the antibodies, polypeptides and proteins of the invention that bind to CK8 are antibodies, polypeptides and proteins that bind preferentially to peptide a) and c). In some embodiments, the antibodies, polypeptides and proteins of the invention that bind to CK8 are antibodies, polypeptides and proteins that bind preferentially to peptide a) and d). In some embodiments, the antibodies, polypeptides and proteins of the invention that bind to CK8 are antibodies, polypeptides and proteins that bind preferentially to peptide a) and e). In some embodiments, the antibodies, polypeptides and proteins of the invention-that bind to CK8 are antibodies, polypeptides and proteins that bind preferentially to peptide a) and f). In some embodiments, the antibodies, polypeptides and proteins of the invention that bind to CK8 are antibodies, polypeptides and proteins that bind preferentially to peptide a) and g). In some embodiments, the antibodies, polypeptides and proteins of the invention that bind to CK8 are antibodies, polypeptides and proteins that bind preferentially to peptide b) and c). In some embodiments, the antibodies, polypeptides and proteins of the invention that bind to CK8 are antibodies, polypeptides and proteins that bind preferentially to peptide b) and d). In some embodiments, the antibodies, polypeptides and proteins of the invention that bind to CK8 are antibodies, polypeptides and proteins that bind preferentially to peptide b) and e). In some embodiments, the antibodies, polypeptides and proteins of the invention that bind to CK8 are antibodies, polypeptides and proteins that bind preferentially to peptide b) and f). In some embodiments, the antibodies, polypeptides and proteins of the invention that bind to CK8 are antibodies, polypeptides and proteins that bind preferentially to peptide b) and g). In some embodiments, the antibodies, polypeptides and proteins of the invention that bind to CK8 are antibodies, polypeptides and proteins that bind preferentially to peptide a), b), and c).

In some embodiments, the antibody of the invention is an antibody mhoe-4 that is produced by a host cell with a deposit number of ATCC No. PTA-3159 or progeny thereof. The present invention also encompasses various formulations of mhoe-4 and equivalent antibodies or polypeptide fragments (e.g., Fab, Fab′, F(ab′)₂, Fv, Fc, etc.), chimeric antibodies, single chain (ScFv), mutants thereof, fusion proteins comprising an antibody portion, humanized antibodies, and any other modified configuration of mhoe-4 that comprises an antigen (Ag-hoe4; CK8), recognition site of the required specificity. The invention also provides human antibodies displaying one or more of the biological characteristics of mhoe-4. The equivalent antibodies of mhoe-4 (including humanized antibodies and human antibodies), polypeptide fragments of mhoe-4, and polypeptides comprising any of these fragments are identified and characterized by any (one or more) of the five criteria described above. In some embodiments, the antibody of the invention is an antibody that preferentially binds to the epitope that mhoe-4 binds preferentially.

Accordingly, the invention provides any of the following (or compositions, including pharmaceutical compositions), comprising any of the following: (a) antibody mhoe-4 produced by the host cell with a deposit number of ATCC No. PTA-3159 or its progeny; (b) a humanized form of antibody mhoe-4; (c) an antibody comprising one or more (at least one, two, three, four, five, or six) of the light chain and/or heavy chain variable regions of antibody mhoe-4; (d) an antibody comprising one or more of the light chain and/or heavy chain CDRs of mhoe-4; (e) an antibody comprising a heavy and/or a light chain of mhoe-4; (f) a chimeric form of antibody mhoe-4; (g) a human antibody that is equivalent to mhoe-4. A humanized form of the antibody may or may not have CDRs identical to mhoe-4, or antibody produced by the host cell with a deposit number of ATCC No. PTA-3159. Determination of CDR regions is well within the skill of the art. In some embodiments, the invention provides an antibody which comprises at least one CDR that is substantially homologous to at least one CDR, at least two, at least three, at least four, at least 5 CDRs of mboe-4 (or, in some embodiments substantially homologous to all 6 CDRs of mhoe-4, or derived from mhoe-4), or antibody produced by the host cell with a deposit number of ATCC No. PTA-3159. Other embodiments include antibodies which have at least two, three, four, five, or six CDR(s) that are substantially homologous to at least two, three, four, five or six CDRs of mhoe-4 or derived from mhoe-4, or antibody produced by the host cell with a deposit number of ATCC No. PTA-3159. It is understood that, for purposes of this invention, binding specificity and/or overall activity (which may be in terms of reducing the proliferation of cancerous cells, inducing apoptotic cell death in the cancer cell, delaying the development of metastasis, and/or treating palliatively) is generally retained, although the extent of activity may vary compared to mhoe-4 (may be greater or lesser). The invention also provides methods of making any of these antibodies. Methods of making antibodies are known in the art and are described herein.

The invention also provides polypeptides comprising an amino acid sequence of the antibodies of the invention, such as mhoe-4. In some embodiments, the polypeptide comprises one or more of the light chain and/or heavy chain variable regions of the antibody mhoe-4. In some embodiments, the polypeptide comprises one or more of the light chain and/or heavy chain CDRs of mhoe-4. In some embodiments, the polypeptide comprises three CDRs of the light chain and/or heavy chain of mhoe-4. In some embodiments, the polypeptide comprises an amino acid sequence of mhoe-4 that has any of the following: at least 5 contiguous amino acids of a sequence of mhoe-4, at least 8 contiguous amino acids, at least about 10 contiguous amino acids, at least about 15 contiguous amino acids, at least about 20 contiguous amino acids, at least about 25 contiguous amino acids, at least about 30 contiguous amino acids, wherein at least 3 of the amino acids are from a variable region of mhoe-4. In one embodiment, the variable region is from a light chain of mhoe-4. In another embodiment, the variable region is from a heavy chain of mhoe-4. In another embodiment, the 5 (or more) contiguous amino acids are from a complementarity determining region (CDR) of mhoe-4.

Antibodies may be polyclonal (e.g., not homogeneous) or monoclonal. Methods of making monoclonal antibodies are known in the art. One method which may be employed is the method of Kohler and Milstein, Nature 256:495-497 (1975) or a modification thereof. In general, a mouse or rat is used for immunization but other animals may also be used. The immunogen can be, but is not limited to, primary cells, cultured cell lines, cancerous cells, nucleic acids, tissue, or peptides. In one embodiment, human fetal ovarian epithelial cells (HOE) are used. Methods for isolating and culturing HOE cells are detailed in the Examples section. Cells used for immunogen, for example, HOE cells, may be cultured for a period of time (at least 24 hours) prior to their use as an immunogen. Cells (e.g., HOE cells) may be used as immunogens by themselves or in combination with a non-denaturing adjuvant, such as Ribi. In general, cells (e.g., HOE cells) should be kept intact and preferably viable when used as immunogens. Intact cells may allow antigens to be detected better than ruptured cells. Use of denaturing or harsh adjuvants, e.g., Freud's adjuvant, may rupture the HOE cells and therefore is discouraged. In another embodiment, full length cytokeratin 8 (CK8) or any fragments of CK8 are used as immunogen. In another embodiment, peptides FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), or EDIANRSRAEAE (SEQ ID NO:7) conjugated with a carrier such as BSA or KLH are used as immunogen. These peptide sequences are contained in CK8. The immunogen may be administered multiple times at periodic intervals such as, bi-weekly, or weekly, or may be administered in such a way as to maintain viability in the animal (e.g., in a tissue recombinant).

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies which bind specifically to the immunogen (e.g., surface of the HOE cells, surface of cancer cell lines, fetal ovary sections, CK8, one or more of the peptides FLEQQNKMLETK (SEQ ID NO: 1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7), etc.) using FACS or immunohistochemistry (IHC screening). The selected monoclonal antibody-secreting hybridomas are then cultured either in vitro (e.g., in tissue culture bottles or hollow fiber reactors), or in vivo (e.g., as ascites in mice). The examples further detail the methods utilized to obtain and screen an antibody mhoe-4, which binds to Ag-hoe4 (cytokeratin 8). Methods of culturing hybridoma under conditions to generate the antibody mhoe-4, and purifying the antibody are known in the art and are further detailed in Examples 2 and 3.

Monoclonal antibody-secreting hybridomas described above can be selected for producing antibodies that bind preferentially to one or more of the peptides FLEQQNKMLETK (SEQ ID NO:1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7). In one embodiment, hybridomas are selected for producing antibodies that bind preferentially to one of the seven peptides. In another embodiment, hybridomas are selected for producing antibodies that bind preferentially to two of the seven peptides. In another embodiment, hybridomas are selected for producing antibodies that bind preferentially to three of the seven peptides. In another embodiment, hybridomas are selected for producing antibodies that bind preferentially to four of the seven peptides. In another embodiment, hybridomas are selected for producing antibodies that bind preferentially to five of the seven peptides. In another embodiment, hybridomas are selected for producing antibodies that bind preferentially to six of the seven peptides. In another embodiment, hybridomas are selected for producing antibodies that bind preferentially to all of the seven peptides.

As another alternative to the cell fusion technique, EBV immortalized B cells may be used to produce monoclonal antibodies of the subject invention. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

In another alternative, the antibodies can be made recombinantly. Methods for making recombinant antibodies are well-known in the art. Monoclonal antibody mhoe-4 and any other equivalent antibodies can be sequenced and produced recombinantly in vitro. In one embodiment, mhoe-4 is sequenced and the polynucleotide sequence is then cloned into a vector for expression or propagation. The sequence encoding the antibody of interest may be maintained in a vector in a host cell and the host cell can then be expanded and frozen for future use. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. (1994) 12:433-455.

The invention includes polypeptides comprising an amino acid sequence of the antibodies of this invention, such as mhoe-4. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, a mhoe-4 polypeptide could be produced by an automated polypeptide synthesizer employing the solid phase method.

The invention also encompasses single chain variable region fragments (“scFv”) of antibodies of this invention, such as mhoe-4. Single chain variable region fragments are made by linking light and/or heavy chain variable regions by using a short linking peptide. Bird et al. (1988) Science 242: 423-426. An example of a linking peptide is (GGGGS)₃ (SEQ ID NO:8), which bridges approximately 3.5 nm between the carboxy terminus of one variable region and the amino terminus of the other variable region. Linkers of other sequences have been designed and used. Bird et al. (1988). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention includes modifications to antibodies, such as antibody mhoe-4, including functionally equivalent antibodies and polypeptides of mhoe-4 which do not significantly affect their properties and variants which have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tryosine. These polypeptides also include glycosylated and nonglycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified mhoe-4 polypeptides are made using established procedures in the art and can be screened using standard assays known in the art, some of which are described below and in the Examples.

The invention also encompasses fusion proteins comprising one or more fragments from the antibodies of this invention, such as mhoe-4. In one embodiment, a fusion polypeptide is provided that comprises at least 10 contiguous amino acids of variable light chain region and at least 10 amino acids of variable heavy chain region. In another embodiment, the fusion polypeptide contains a heterologous immunoglobulin constant region. In another embodiment, the fusion polypeptide contains a light chain variable region and a heavy chain variable region of mhoe-4. For purposes of this invention, a mhoe-4 fusion protein contains one or more mhoe-4 polypeptides and another amino acid sequence to which it is not attached in the native molecule, for example, a heterologous sequence or a homologous sequence from another region. A mhoe-4 polypeptide can be created by methods known in the art, for example, synthetically or recombinantly.

In another embodiment, mhoe-4 chimeras are provided in which the heavy and/or light chains are fusion proteins. In some embodiments, the constant domain of the chains is from one particular species and/or class, and the variable domains are from a different species and/or class. For instance, a “humanized” mhoe-4 antibody (in some embodiments) is one in which the constant region is of human origin, and the variable region is from mhoe-4 (i.e., murine). Also embodied within the invention is an antibody with a humanized variable region, in which (in some embodiments) the CDR-regions-comprise mhoe-4 amino acid sequences, while the framework regions are derived from human sequences. Other forms of humanized antibodies are known in the art and described herein. Also embodied are functional fragments of chimeras. An example is a humanized Fab fragment, which contains a human hinge region, a human first constant region, a human kappa light or heavy chain constant region, and the variable region of light and/or heavy chain from mhoe-4. The humanized mhoe-4 Fab fragments can in turn be made to form Fab dimers. Typically, the mhoe-4 fusion proteins and mhoe-4 chimeras of this invention are made by preparing an expressing a polynucleotide encoding them using recombinant methods described herein, although they may also be prepared by other means known in the art, including, for example, chemical synthesis.

The invention also encompasses humanized antibodies. The polynucleotide sequence of an antibody, such as mhoe-4 or other equivalent antibodies may be used for genetic manipulation to generate a “humanized” antibody, or to improve the affinity, or other characteristics of the antibody. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. For example, the constant region may be engineered to more resemble human constant regions to avoid immune response if the antibody is used in clinical trials and treatments in humans. See, for example, U.S. Pat. Nos. 5,997,867 and 5,866,692.

A number of “humanized” antibody molecules comprising an antigen-binding site derived from a non-human immunoglobulin have been described, including chimeric antibodies having rodent V regions and their associated complementarity determining regions (CDRs) fused to human constant domains. See, for example, Winter et al. Nature 349:293-299 (1991); Lobuglio et al. Proc. Nat. Acad. Sci. USA 86:4220-4224 (1989); Shaw et al. J. Immunol. 138:4534-4538 (1987); and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988); Verhoeyen et al. Science 239:1534-1536 (1988); and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent antihuman antibody molecules which limits the duration and effectiveness of therapeutic applications of those moieties in human recipients. Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; 6,350,861; and PCT WO 01/27160.

In yet another alternative, fully human antibodies may be obtained by using commercially available mice which have been engineered to express specific human immunoglobulin proteins. Transgenic animals which are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

This invention also provides compositions comprising mhoe-4 or mhoe-4 equivalent antibodies or polypeptides conjugated (for example, linked) to a therapeutic agent, such as a radioactive molecule, a toxin (e.g., calicheamicin), or a chemotherapeutic molecule, or to liposomes or other vesicles containing chemotherapeutic compounds. The compositions, when administered to an individual, can target these agents to a cancer cell expressing cytokeratin 8 recognized by the antibody or polypeptide(s) and thus can, for example, eliminate cancerous cells and/or suppress proliferation and/or growth of cancerous cells. For simplicity, reference will be made generally to mhoe-4 or antibodies with the understanding that these methods apply to any of the CK8 binding embodiments described herein. These, conjugation generally refers to linking these components as described herein. The linking (which is generally fixing these components in proximate association at least for administration) can be achieved in any number of ways, as described below.

A radioactive molecule of this invention includes any radioisotope which is effective in destroying a cancerous cell. Examples include, but not limited to, cobalt-60 and X-rays. Additionally, naturally occurring radioactive elements such as uranium, radium, and thorium which typically represent mixtures of radioisotopes, are suitable examples of a radioactive molecule.

A toxin of the invention include, but not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. The antibodies of the invention can be internalized within the carcinoma cells to which they bind and are therefore particularly useful for therapeutic applications, for example, delivering into the cells toxins that need to be internalized for their adverse activity. Examples of such toxins include, but not limited to, saporin, calicheamicin, and maytansinoid.

The antibodies or polypeptides of the invention can be conjugated (linked) to a radioactive molecule, a toxin, or other therapeutic agents, or to liposomes or other vesicles containing therapeutic agents covalently or non-covalently, directly or indirectly. The antibody may be linked to the radioactive molecule, the toxin, or the therapeutic molecule at any location along the antibody so long as the antibody is able to bind its target CK8.

A toxin or a therapeutic agent may be coupled (e.g., covalently bonded) to a suitable monoclonal antibody either directly or indirectly (e.g., via a linker group, or, alternatively, via a linking molecule with appropriate attachment sites, such as a platform molecule as described in U.S. Pat. No. 5,552,391). The toxin and therapeutic agent of the present invention can be coupled directly to the particular targeting proteins using methods known in the art. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies or polypeptides can also be linked to a therapeutic agent via a microcarrier. Microcarrier refers to a biodegradable or a non-biodegradable particle which is insoluble in water and which has a size of less than about 150, 120 or 100 μm in size, more commonly less than about 50-60 μm, preferably less than about 10, 5, 2.5, 2 or 1.5 μm. Microcarriers include “nanocarriers”, which are microcarriers have a size of less than about 1 μm, preferably less than about 500 nm. Such particles are known in the art. Solid phase microcarriers may be particles formed from biocompatible naturally occurring polymers, synthetic polymers or synthetic copolymers, which may include or exclude microcarriers formed from agarose or cross-linked agarose, as well as other biodegradable materials known in the art. Biodegradable solid phase microcarriers may be formed from polymers which are degradable (e.g., poly(lactic acid), poly(glycolic acid) and copolymers thereof) or erodible (e.g., poly(ortho esters such as 3,9-diethylidene-2,4,8,10-tetraoxaspiro[5.5]undecane (DETOSU) or poly(anhydrides), such as poly(anhydrides) of sebacic acid) under mammalian physiological conditions. Microcarriers may also be liquid phase (e.g., oil or lipid based), such liposomes, iscoms (immune-stimulating complexes, which are stable complexes of cholesterol, and phospholipid, adjuvant-active saponin) without antigen, or droplets or micelles found in oil-in-water or water-in-oil emulsions, provided the liquid phase microcarriers are biodegradable. Biodegradable liquid phase microcarriers typically incorporate a biodegradable oil, a number of which are known in the art, including squalene and vegetable oils. Microcarriers are typically spherical in shape, but microcarriers which deviate from spherical shape are also acceptable (e.g., elipsoid, rod-shaped, etc.). Due to their insoluble nature (with respect to water), microcarriers are filterable from water and water-based (aqueous) solutions.

The antibody or polypeptide conjugates of the present invention may include a bifunctional linker which contains both a group capable of coupling to a toxic agent or therapeutic agent and a group capable of coupling to the antibody. A linker can function as a spacer to distance an antibody from an agent in order to avoid interference with binding capabilities. A linker can be cleavable or non-cleavable. A linker can also serve to increase the chemical reactivity of a substituent on an agent or an antibody, and thus increase the coupling efficiency. An increase in chemical reactivity may also facilitate the use of agents, or functional groups on agents, which otherwise would not be possible. The bifunctional linker can be coupled to the antibody by means which are known in the art. For example, a linker containing an active ester moiety, such as an N-hydroxysuccinimide ester, can be used for coupling to lysine residues in the antibody via an amide linkage. In another example, a linker containing a nucleophilic amine or hydrazine residue can be coupled to aldehyde groups produced by glycolytic oxidation of antibody carbohydrate residues. In addition to these direct methods of coupling, the linker can be indirectly coupled to the antibody by means of an intermediate carrier such as an aminodextran. In these embodiments the modified linkage is via either lysine, carbohydrate, or an intermediate carrier. In one embodiment, the linker is coupled site-selectively to free thiol residues in the protein. Moieties which are suitable for selective coupling to thiol groups on proteins are well known in the art. Examples include disulfide compounds, α-halocarbonyl and α-halocarboxyl compounds, and maleimides. When a nucleophilic amine function is present in the same molecule as an α-halo carbonyl or carboxyl group the potential exists for cyclization to occur via intramolecular alkylation of the amine. Methods to prevent this problem are well known to one of ordinary skill in the art, for example by preparation of molecules in which the amine and α-halo functions are separated by inflexible groups, such as aryl groups or trans-alkenes, that make the undesired cyclization stereochemically disfavored. See, for example, U.S. Pat. No. 6,441,163 for preparation of conjugates of maytansinoids and antibody via a disulfide moiety.

One of the cleavable linkers that can be used for the preparation of antibody-drug conjugates is an acid-labile linker based on cis-aconitic acid that takes advantage of the acidic environment of different intracellular compartments such as the endosomes encountered during receptor mediated endocytosis and the lysosomes. See, for example, Shen et al., Biochem. Biophys. Res. Commun. 102:1048-1054 (1981) for the preparation of conjugates of daunorubicin with macromolecular carriers; Yang et al., J. Natl. Canc. Inst. 80:1154-1159 (1988) for the preparation of conjugates of daunorubicin to an anti-melanoma antibody; Dillman et al., Cancer Res. 48:6097-6102 (1988) for using an acid-labile linker in a similar fashion to prepare conjugates of daunorubicin with an anti-T cell antibody; Trouet et al., Proc. Natl. Acad. Sci. 79:626-629 (1982) for linking daunorubicin to an antibody via a peptide spacer arm.

An antibody (or polypeptide) of this invention may be conjugated (linked) to a radioactive molecule by any method known to the art. For a discussion of methods for radiolabeling antibody see “Cancer Therapy with Monoclonal AntibodiesT”, D. M. Goldenberg ed. (CRC Press, Boca Raton, 1995).

An antibody (or polypeptide) of this invention may be linked to a labeling agent (alternatively termed “label”) such as a fluorescent molecule, a radioactive molecule or any others labels known in the art. Labels are known in the art which generally provide (either directly or indirectly) a signal.

The ability of the antibodies, polypeptides and proteins of this invention, such as ability to inhibit growth of cancerous cells expressing CK8, ability to delay development of metastasis in an individual with cancer expressing CK8, ability to deliver a therapeutic agent, such as a toxin, or a radioactive compound, to cancerous cells expressing CK8, including ability to deliver a therapeutic agent into cancerous cells expressing CK8, may be tested using methods known in the art, some of which are described in the Examples.

The invention also provides compositions (including pharmaceutical compositions) comprising antibody mhoe-4 or mhoe-4 equivalent antibodies (which, as this disclosure makes clear, include all of the antibodies described herein) or polypeptides and a therapeutic agent.

Methods for Screening Monoclonal Antibodies

Several methods may be used to screen monoclonal antibodies that bind to Ag-hoe4 (cytokeratin 8). One method which may be employed is immunohistochemistry (IHC). Standard immunohistochemical techniques are known to those of average skill in the art. See, for example, Animal Cell Culture Methods (J. P. Mather and D. Barnes, eds., Academic Press, Vol. 57, Ch. 18 and 19, pp. 314-350, 1998). Biological samples (e.g., tissues) may be obtained from biopsies, autopsies, or necropsies. To ascertain if Ag-hoe4 (cytokeratin 8) is present only on cancerous cells, mhoe-4 may be used to detect the presence of Ag-hoe4 (cytokeratin 8) on tissues from individuals with cancer while other non-cancerous tissues from the individual suffering from cancer or tissues from individuals without cancer are used as a control. The tissue can be embedded in a solid or semi-solid substance which prevents damage during freezing (e.g., agarose gel or OCT) and then sectioned for staining. Cancers from different organs and at different grades can be used to screen monoclonal antibodies. Examples of tissues which may be used for screening purposes include but are not limited to ovary, breast, lung, prostate, colon, kidney, skin, thyroid, brain, heart, liver, stomach, nerve, blood vessels, bone, upper digestive tract, and pancreas. Examples of different cancer types which may be used for screening purposes include but are not limited to carcinomas, adenocarcinomas, sarcomas, adenosarcomas, lymphomas, and leukemias.

In yet another alternative, cancerous cells lines such as SK-OV-3 (ATCC #HTB 77), OVCAR-3 (ATCC #HTB 161), Caov-3 (ATCC #HTB 75), LNCaP (ATCC #CRL-1740), COLO 205 (ATCC #CCL 222), A549 (ATCC #CCL 185), PANC-1 (ATCC #CRL 1469), SK-BR-3 (ATCC #HTB 30), SK-MES-1 (ATCC #HTB 58), HT-29 (HTB-38), H9 (ATCC #HTB-176), SW 480 (ATCC #CCL 228), AsPC-1 (ATCC #CRL 1682), Capan-1 (ATCC #HTB 79), CFPAC-1 (ATCC #CRL 1918), HPAF-II (ATCC #CRL-1997), HS-700T (ATCC #HTB 147), ES-2 (ATCC #CRL-1978), and PC-3 (ATCC #CRL 1435) and normal cells from their respective tissues may be used to screen for monoclonal antibodies which are specific for cancerous tissue. Primary, or low passage, cell cultures derived from normal tissues from different organs, including but not limited to, ovary, breast, lung, prostate, colon, kidney, skin, thyroid, aortic smooth muscle, and endothelial cells can be used as negative controls. The cancerous or non-cancerous cells can be grown on glass slides or coverslips, or on plastic surfaces, or prepared in a CellArray™, as described in WO 01/43869, and screened for the binding of antibody using IHC as described above for tissues. Alternatively, cells may be removed from the growth surface using non-proteolytic means and spun into a pellet which is then embedded and treated as tissues for IHC analysis as described above. In another alternative, single cells may be screened by incubating with the primary antibody, a secondary “reporter” antibody linked to a fluorescent molecule and then analyzed using a fluorescent activated cell sorting (FACS) machine.

Several different detection systems may be utilized to detect binding of antibodies to tissue section. Typically, immunohistochemistry involves the binding of a primary antibody to the tissue and then a secondary antibody reactive against the species from the primary antibody was generated and conjugated to a detectable marker (e.g., horseradish peroxidase, HRP, or diaminobenzedine, DAB). One alternative method that may be used is polyclonal mirror image complementary antibodies or polyMICA. PolyMICA (polyclonal Mirror Image Complementary Antibodies) technique, described by D. C. Mangham and P. G. Isaacson (Histopathology (1999) 35(2):129-33), can be used to test binding of primary antibodies (e.g., mhoe-4) to normal and cancerous tissue. Several kinds of polyMICA™ Detection kits are commercially available from The Binding Site Limited (P.O. Box 4073 Birmingham B29 6AT England). Product No. HK004.D is a polyMICA™ Detection kit which uses DAB chromagen. Product No. HK004.A is a polyMICA™ Detection kit which uses AEC chromagen. Alternatively, the primary antibody may be directly labeled with the detectable marker.

The first step in IHC screening to select for an appropriate antibody is the binding of primary antibodies raised in mice (e.g., mhoe-4) to one or more immunogens (e.g., cells or tissue samples). In one embodiment, the tissue sample is sections of frozen tissue from different organs. The cells or tissue samples can be either cancerous or non-cancerous.

Frozen tissues can be prepared, sectioned, with or without fixation, and IHC performed by any of a number of methods known to one familiar with the art. See, for example, Stephan et al. Dev. Biol. 212: 264-277 (1999), and Stephan et al. Endocrinology 140: 5841-54 (1999).

Monoclonal antibodies that are cross-reactive with human cells and that bind to cancerous cells or tissues, but not to normal cells or tissues to the same degree, are selected. Monoclonal antibodies that bind to antigens expressed on one or more cancer types but not to normal cells are also selected. mhoe-4 is an example of an antibody that binds to an antigen present on a number of different cancers, but has limited binding to normal tissues. In accordance with the Budapest Treaty, the hybridoma which produces mhoe-4 has been deposited in the American Type Culture Collection (ATCC) 10801 University Blvd., Manassas Va. 20110-2209 on Mar. 6, 2001 with a Patent Deposit Designation of PTA-3159.

Epitope mapping may be used to further characterize the antibody. Commercially available services (e.g., Pepscan Systems, P.O. Box 2098, 8203 AB Lelystad, The Netherlands) may be used to determine the epitope(s) on the antigen to which an antibody, such as mhoe-4, binds. Using such commercially available resources, mhoe-4 was found to bind to multiple epitopes, with highest affinity preferential binding to FLEQQNKMLETK (SEQ ID NO:1). See Example 7. In addition, specific but lesser affinity binding was detected to the following sequences: QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), EDIANRSRAEAE (SEQ ID NO:7).

Characterization of mhoe-4 Antigen

The antigen for mhoe-4, Ag-hoe4, was identified in one aspect by Western blotting with cell lysates from various human cancers and monoclonal antibody mhoe-4, the hybridoma producing this antibody was deposited at the American Type Culture Collection (ATCC) 10801 University Blvd., Manassas Va. 20110-2209 on Mar. 6, 2001 with a Patent Deposit Designation of PTA-3159. As is known to one of skill in the art, Western blotting can involve running cell lysates and/or cell fractions on a denaturing or non-denaturing gel, transferring the proteins to nitrocellulose paper, and then probing the blot with an antibody (e.g., mhoe-4) to see which proteins are bound by the antibody. This procedure is detailed further in Example 1.

Another method that was used to characterize antigens to which mhoe-4 bind is mass spectrometry analysis. Several types of mass spectrometry analysis may be performed. In one approach, the masses of a tryptic digest of the protein were measured by matrix assisted laser desorption/ionization (MALDI) time-of-flight mass spectrometry (MALDI-TOF-MS) and the resulting list of peptide masses were used as a “fingerprint” of the protein in sequence database searches. In matrix assisted laser desorption/ionization (MALDI), the peptides are co-crystallized with a large excess of a light absorbing matrix. Irradiation of the crystals by a pulsed laser beam results in the rapid sublimation of matrix and the embedded peptide molecules and the generation of intact gas phase ions. For peptides, protonated, singly charged molecular ions are usually formed. The mass/charge ratio (m/z) is measured at high mass accuracy time-of-flight analysis, optionally employing delayed-extraction and/or a reflectron. The retrieved sequences are evaluated by mass analysis of the peptides, matching the peptide masses in the MALDI spectrum after accounting for common modifications such as oxidation, acrylamidation of cysteine and missed cleavages and the use of secondary information (apparent isoelectric point and/or molecular weight). If any ambiguity about the identification by MALDI-TOF-MS still exists, the results can be verified by mass spectrometric peptide sequencing. These and other procedures for using mass spectroscopy to identify known proteins are reviewed in Use of Mass Spectrometry to Study Signaling Pathways (A. Pandey, J. S. Andersen, and M. Mann; 2000).

The surface antigen recognized by a monoclonal antibody of the present invention is first isolated by any method described above and alternatively, can be isolated by any methods known to the average skilled artisan. The protein band which is bound by the antibody was characterized by first digesting the purified protein with a protease, which results in a mixture of peptides. The peptides was then analyzed by MALDI mass spectroscopy and the mass spectrometry pattern is compared to patterns of other proteins. In this manner, the antigen for mhoe-4, Ag-hoe4, was determined to be cytokeratin 8 (CK8). In addition, mhoe-4 was shown to bind to CK8 but not cytokeratin 18 (CK18) by Western blotting.

In another alternative, the antigen or protein of interest may be subjected to sequencing by Edman degradation, which is well-known to those of skill in the art. The peptide information generated from mass spectrometry or Edman degradation can be used to design probes or primers that are used to clone the antigen of interest.

Ag-hoe4 can be further characterized by its location within a cell. Without being bound by theory, Ag-hoe4 (cytokeratin 8) is a cell-associated antigen that is expressed at least on the surface of a cell. Since the method of generating monoclonal antibody mhoe-4 involved using intact cells as immunogen, the monoclonal antibody that was generated was most likely against an antigenic determinant on the surface of the cell. Such cell surface proteins may, however, also be present inside the cell, or secreted or released from the cell surface, in addition to being present on the cell surface. Whether the antigen is present on the cell surface or interior or released from the cell may differ depending on the type of cells, or alternatively may depend on the different stages of the cell cycle, different developmental stages, or in diseased compared to non-diseased (i.e., normal), cells.

Further characterization of antigen was accomplished by determining expression patterns on different tissues or cells, copy number on cells and/or tissues, and by the antibodies which bind to it. In one aspect, the expression patterns was determined by using immunohistochemical techniques with biological samples. The expression pattern of the antigen can be assessed in individuals with and without cancer or alternatively another disease state. Copy number of antigens can be determined by using standard Scatchard analysis. Determining expression patterns of Ag-hoe4 was described in further detail in Examples 8-10.

Methods of Diagnosing Cancer Using mhoe-4, mhoe-4 Equivalent Antibodies or Polypeptides which Bind to CK8

Monoclonal antibody mhoe-4 and equivalent antibodies or polypeptide derivatives of mhoe-4 which bind CK8 made by the methods disclosed herein may be used to identify or detect the presence or absence of cancerous cells in a variety of tissues, including but not limited to, ovary, breast, lung, prostate, colon, kidney, skin, thyroid, brain, heart, liver, stomach, nerve, blood vessels, bone, upper digestive tract, and pancreas for purposes of diagnosis. For simplicity, reference will be made generally to mhoe-4 or antibodies with the understanding that these methods apply to any of the CK8 binding embodiments described herein. Detection generally involves contacting cells with an antibody or a polypeptide described herein that binds to CK8 and the formation of a complex between Ag-hoe4 (cytokeratin 8) and an antibody (e.g., mhoe-4, a humanized antibody of mhoe-4, a human antibody or any other CK8 binding moiety) which binds specifically to Ag-hoe4 (cytokeratin 8). The formation of such a complex can be in vitro or in vivo. Without being bound by theory, monoclonal antibody mhoe-4 can bind to Ag-hoe4 (cytokeratin 8) through the Ag-hoe4 (cytokeratin 8) expressed on the surface of cells.

In some embodiments, methods are provided for detecting presence or absence of thyroid cancerous cells by detecting CK8 from cells. CK8 from thyroid cells can be detected using any method, including but not limited to detection of CK8 mRNA, and detection of CK8 protein. Any CK8 binding moiety can be used, such as those described herein (e.g., mhoe-4, mhoe-4 equivalent antibodies such as those which bind the same epitope as mhoe-4). As used herein, detection may include qualitative and/or quantitative detection and may include comparing the level measured to a normal thyroid cell for an increased level of expression of CK8 in cancerous cells.

One method of using the antibodies for diagnosis is in vivo tumor imaging by linking the antibody to a labeling moiety (e.g., a fluorescent agent, a radioactive or radioopaque agent), administering the antibody to the patient and using an x-ray or other imaging machine to visualize the localization of the labeled antibody at the surface of cancer cells expressing the antigen. Labeling moieties are known in the art.

In other methods, the cancerous cells are removed and the tissue prepared for immunohistochemistry by methods well known in the art (e.g., embedding in a freezing compound, freezing and sectioning, with or without fixation; fixation and paraffin embedding with or without various methods of antigen retrieval and counterstaining). The monoclonal antibodies may also be used to identify neoplasms at different stages of development. The antibodies may also be used to determine which patients' tumors express the antigen on their surface at a pre-determined level and are thus candidates for immunotherapy using antibodies directed against said antigen.

Antibodies (or polypeptides) recognizing the antigen may also be used to create diagnostic immunoassays for detecting antigen released or secreted from living or dying cancer cells in bodily fluids, including but not limited to, blood, saliva, urine, pulmonary fluid, or ascites fluid. As discussed in further detail in the Examples, mhoe-4 can bind to adenocarcinomas, carcinomas, sarcomas, or adenosarcomas from tissues including but not limited to ovary, breast, lung, prostate, colon, kidney, liver, thyroid, upper digestive tract, and pancreas. Methods of using mhoe-4 for diagnostic purposes is useful both before and after any form of anti-cancer treatment, e.g., chemotherapy or radiation therapy, to determine which tumors are most likely to respond to a given treatment, patient prognosis, tumor subtype or origin of metastatic disease, and progression of the disease or response to treatment.

Methods of using mhoe-4, mhoe-4 Equivalent Antibodies or Polypeptides for Therapeutic Purposes

Monoclonal antibody mhoe-4 and equivalent antibodies made by the methods disclosed herein may be used for therapeutic purposes in individuals with cancer, including but not limited to cancer of the ovary, breast, lung, prostate, colon, kidney, liver, thyroid, upper digestive tract, or pancreas. These therapeutic methods also apply to the linked embodiments described above. For simplicity, reference will be made generally to mhoe-4 or antibodies with the understanding that these methods apply to any of the CK8 binding embodiments as well as humanized antibodies and human antibodies described herein including linked embodiments. Therapy with mhoe-4 can involve formation of complexes of mhoe-4 and Ag-hoe4 (CK8) both in vitro and/or in vivo as described above. In one embodiment, monoclonal antibody mhoe-4 can bind to and reduce the proliferation of cancerous cells (e.g., prostate cancer cells or ovarian cancer cells). In another embodiment, monoclonal antibody mhoe-4 can bind to and induce apoptotic cell death in the cancer cell. In another embodiment, monoclonal antibody mhoe-4 can bind to cancerous cells and delay the development of metastasis. In another embodiment, monoclonal antibody mhoe-4 can bind to cancerous cells and deliver a therapeutic agent (such as a toxin, or a radioactive compound) linked to mhoe-4 to cancerous cells. For some embodiments, therapeutic agent (such as a toxin) is introduced into a cell (i.e., is internalized). Particularly suitable agents for these methods include agents which are active inside the cell. Examples of such agents include but not limited to saporin, calicheamicin, and maytansinoid. In some embodiments, these agents are linked to mhoe-4 and are internalized in prostate cancer cells. In yet another embodiment, an individual with cancer is given palliative treatment with mhoe-4. Palliative treatment of a cancer patient involves treating or lessening the adverse symptoms of the disease, or iatrogenic symptoms resulting from other treatments given for the disease without directly affecting the cancer progression. This includes treatments for easing of pain, nutritional support, sexual problems, psychological distress, depression, fatigue, psychiatric disorders, nausea, vomiting, etc.

This invention also provides methods of inhibiting growth and/or proliferation of cancer cells (e.g., prostate cancer cells or ovarian cancer cells) using an antibody that binds to cytokeratin 8. Other antibodies that bind to cytokeratin 8 are known in the art, for example, antibody C-OU1 described in U.S. Pat. No. 5,338,661, and antibodies described in U.S. Pat. No. 4,775,620. The method of testing activity of an antibody in inhibiting growth and/or proliferation of cancer cells are known in the art and are described in detail in Examples 11-13.

In yet another embodiment, mhoe-4 can bind to CK8 expressing cancerous cells and induce an active immune response against the cancerous cells expressing Ag-hoe4 (cytokeratin 8). In some cases, the active immune response can cause the death of the cancerous cells (e.g., mhoe-4 binding to cancer cells inducing apoptotic cell death), or inhibit the growth (e.g., block cells cycle progression) of the cancerous cells. In other cases, mhoe-4 can bind to cancerous cells and antibody dependent cellular cytotoxicity (ADCC) can eliminate cancerous cells to which mhoe-4 binds. Accordingly, the invention provides methods of stimulating an immune response comprising administering any of the compositions described herein.

In some cases, mhoe-4 binding can also activate both cellular and humoral immune responses and recruit more natural killer cells or increased production of cytokines (e.g., IL-2, IFN-γ, IL-12, TNF-α, TNF-β, etc.) that further activate an individual's immune system to destroy cancerous cells. In yet another embodiment, mhoe-4 can bind to cancerous cells and macrophages or other phagocytic cell can opsonize the cancerous cells.

In some embodiments, the invention provides methods of conferring passive immunity comprising administering any of the compositions described herein.

The invention provides methods of delivering any of the compositions (including conjugates) described herein to a CK8 expressing cell, such as a CK8 expression cancer cells. These methods entail administering the compositions (including conjugates) described herein to an individual. In some embodiments, the methods provide for introducing, for example, a conjugate into a target cell. In yet another embodiment, antibody mhoe-4 can be conjugated to a therapeutic agent (such as a radioactive molecule or a toxin) or to liposomes or other vesicles containing therapeutic agents and administered to an individual to target these agents to the cancer cell containing the antigen recognized by the antibody and thus eliminate cancerous cells. In yet another embodiment, the antibody can be employed as adjuvant therapy at the time of the surgical removal of a cancer expressing the antigen in order to delay the development of metastasis. The antibody can also be administered before surgery (neoadjuvant therapy) in a patient with a tumor expressing the antigen in order to decrease the size of the tumor and thus enable or simplify surgery, spare tissue during surgery, and/or decrease the resulting disfigurement.

Various formulations of mhoe-4 and equivalent antibodies or fragments (e.g., Fab, Fab′, F(ab′)₂, Fv, Fc, etc.), such as chimeric antibodies, single chain (ScFv), mutants thereof, fusion proteins comprising an antibody portion, humanized antibodies, and any other modified configuration of mhoe-4 that comprises an antigen (Ag-hoe4) recognition site of the required specificity, may be used for administration. In some embodiments, mhoe-4 antibodies or various formulations of mhoe-4 thereof may be administered neat. In other embodiments, mhoe-4 or various formulations of mhoe-4 (including any composition embodiment described herein) thereof and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intramuscularly, etc.), although other forms of administration (e.g., oral, mucosal, etc) can be also used. Accordingly, mhoe-4 antibody and equivalents thereof are preferably combined with pharmaceutically acceptable vehicles such as saline, Ringer's solution, dextrose solution, and the like. The particular dosage regimen, i.e., dose, timing and repetition, will depend on the particular individual and that individual's medical history. Generally, a dose of at least about 1 ug/kg body weight, more preferably at least about 10 ug/kg body weight, even more preferably at least about 50 ug/kg body weight, even more preferably at least about 100 ug/kg body weight, even more preferably at least about 250 ug/kg body weight, even more preferably at least about 500 ug/kg body weight, even more preferably at least about 750 ug/kg body weight, even more preferably at least about 1 mg/kg body weight, even more preferably at least about 3 mg/kg body weight, even more preferably at least about 5 mg/kg body weight, even more preferably at least about 10 mg/kg body weight, or more, is administered. Empirical considerations, such as the half-life, generally will contribute to determination of the dosage. Antibodies which are compatible with the human immune system, such as humanized antibodies or fully human antibodies, may be used to prolong half-life of the antibody and to prevent the antibody being attacked by the host's immune system.

In some individuals, more than one dose may be required. Frequency of administration may be determined and adjusted over the course of therapy, and is based on reducing the number of cancerous cells, maintaining the reduction of cancerous cells, reducing the proliferation of cancerous cells, or delaying the development of metastasis. The presence of cancerous cells can be identified by any number of methods known to one of skill in the art or discussed herein (e.g., detection by immunohistochemistry or flow cytometry of biopsies or biological samples). In some cases, sustained continuous release formulations of mhoe-4 antibodies may be appropriate. Various formulations and devices for achieving sustained release are known in the art.

In one embodiment, dosages for mhoe-4 antibodies may be determined empirically in individuals who have been given one or more administration(s). Individuals are given incremental dosages of mhoe-4. To assess efficacy of mhoe-4 or other equivalent antibody, markers of the specific cancer disease state can be monitored. These markers include: direct measurements of tumor size via palpation or visual observation; indirect measurement of tumor size by x-ray or other imaging techniques; an improvement as assessed by direct tumor biopsy and microscopic examination of the tumor sample; the measurement of an indirect tumor marker (e.g., PSA for prostate cancer), a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In some embodiments, more than one antibody may be present. The antibodies can be monoclonal or polyclonal. Such compositions may contain at least one, at least two, at least three, at least four, at least five different antibodies that are reactive against carcinomas, adenocarcinomas, sarcomas, or adenosarcomas. mhoe-4 antibody can be admixed with one or more antibodies reactive against carcinomas, adenocarcinomas, sarcomas, or adenosarcomas in organs including but not limited to ovary, breast, lung, prostate, colon, kidney, skin, thyroid, bone, upper digestive tract, and pancreas. A mixture of antibodies, as they are often denoted in the art, may be particularly useful in treating a broader range of population of individuals.

Kits Comprising Antibodies and Polypeptides of the Invention which Bind to Ag-hoe4 (Cytokeratin 8)

The invention also provides kits comprising antibodies or any of the compositions described herein which bind to cytokeratin 8 for use in diagnosis or therapy. Accordingly, the kits comprise an antibody which can bind to cytokeratin 8 preferentially and/or form a complex with cytokeratin 8 (useful, for example, for detecting thyroid cancerous cells). In some embodiments, the kits comprise antibody mhoe-4 or an antibody that preferentially binds to the same epitope as mhoe-4 preferentially binds. In some embodiments, the kits comprise antibody mhoe-4 or an antibody that preferentially binds to the same epitope as mhoe-4 (such as SEQ ID NO: 1) preferentially binds linked to a therapeutic agent or a labeling agent. In some embodiments, the kits comprise antibody mhoe-4 or an antibody that preferentially binds to the same epitope as mhoe-4 (such as SEQ ID NO:1) preferentially binds and a therapeutic agent or a labeling agent. These kits may further include instruction and/or reagent for linking the antibody or any antibody or polypeptide embodiments described herein to the therapeutic agent(s) or the labeling agent(s). In some aspects, the binding of an antibody (e.g., monoclonal, polyclonal, human, humanized, etc.) to cytokeratin 8 is used for diagnosing cancer in an individual, for example, kits for detecting presence or absence of cancerous cells, and kits for detecting presence or absence of thyroid cancerous cells. In other aspects, the kits may be used, for example, to treat an individual with cancer or a family history of cancer. Kits for treating individual with cancer include but not limited to kits for inhibiting growth and/or proliferation of cancer cells, for delivering a therapeutic agent to cancerous cells, for delivering a therapeutic agent into cancerous cells such as prostate. The kits of this invention are in suitable packaging, and may optionally provide additional components such as, buffers and instructions for determining binding to Ag-hoe4 (cytokeratin 8), such as capture reagents, developing reagents, labels, reacting surfaces, means for detection, control samples, and interpretive information. The instructions may be for any measurement of antigen binding, including, but not limited to, those assays described herein. In other embodiments, the instructions may be for any of the methods described herein, including: instructions for inhibiting grow and/or proliferation of cancerous cells, for delivering a therapeutic agent to cancerous cells, for delivering a therapeutic agent into cancerous cells such as prostate. In some embodiments, reagents described above are supplied such that multiple measurements may be made, such as allowing for measurements in the same individual over time or multiple individuals. Any appropriate means for detecting binding of the antibodies may be employed (and provided in the kits) such as a labeled anti-human antibody, wherein the label may be an enzyme, fluorophore, chemiluminescent material radioisotope or coenzyme. Generally, the label used will be an enzyme.

The following examples are provided to illustrate, but not to limit, the invention.

### EXAMPLES

### Example 1

### Preparation of Human Ovarian Epithelial Cells as an Immunogen

Human fetal ovaries of gestational age between 17 to 25 weeks were obtained from Advanced Bioscience Research at Alameda County, Calif. Ovaries were procured and shipped to the lab in tissue culture medium under wet ice bath. Immediately upon arrival, the ovaries were cleaned of excess connective tissues, carefully separated from fallopian tube, and washed five times with fresh tissue culture medium.

The ovaries were minced with scissors or cut into small pieces (less than 1 mm thick) with a razor blade. The tissue pieces from each ovary were plated directly in a T75 flask freshly coated with laminin with 10 ml preferred nutrient medium as disclosed herein. Further dissociation of the ovaries with collagenase-dispase (0.5%) for 30 minutes at 37° C. could be done, but the procedure reduced the recovery of HOE cells. The cells were cultured in F12/DMEM supplemented with 10 μg/ml insulin, 10 nM recombinant human heregulin β1, 10 ng/ml epidermal growth factor, and 2% fetal bovine serum (by volume) in T-75 flasks or 100 mm plates at standard incubation conditions. Under these culture conditions, the human ovarian epithelial cells (HOE) attached to the plastic of the tissue culture container and grew as a monolayer. Cultures were passaged by first typsinizing the cells to detach them from the tissue culture container and then re-plating the cells in the same culture medium at a 1 to 5 split ratio every 5 to 7 days. The cells were washed with F12/DMEM and grown in serum free medium (e.g., F12/DMEM plus 10 μg/ml insulin, 10 nM recombinant human heregulin β1, 10 ng/ml epidermal growth factor) for a minimum of 24 hour prior to harvesting for injections.

To harvest the cells, the cells were rinsed once with calcium and magnesium free Hanks saline solution, incubated in 0.02% EDTA in Hanks saline solution at 37° C. for 15 minutes. The cells were detached from the culture surface by gentle tapping. The cell suspension was precipitated by centrifuge at 1000 rpm for 10 minutes. The supernatant was removed and cells were resuspended in serum free medium (F12/DMEM) containing appropriate non-denaturing adjuvant.

### Example 2

### Generation of Monoclonal Antibodies Against HOE

Approximately 10⁶ HOE cells per mouse were injected into Balb/c mice via foot-pad, once a week. Non-denaturing adjuvants, (e.g., 200 μl Ribi was used to 100 μl of cell suspension) were used. After 6 weeks of weekly injection, a drop of blood were drawn from the tail of each immunized animal to test the titer of antibodies against HOE using FACS analysis. When the titer reached at least 1:2000, the mice were sacrificed in a CO₂ chamber followed by cervical dislocation. Lymph nodes were harvested for hybridoma preparation.

Lymphocytes from mice with the highest titer were fused with the mouse myeloma line X63-Ag8.653 using 35% polyethylene glycol 4000. On day 10 following the fusion, the hybridoma supernatants were screened for the presence of HOE-specific monoclonal antibodies by fluorescence activated cell sorting (FACS). Conditioned medium from each hybridoma was incubated for 30 minutes with an aliquot of HOE cells, or cells from cultures of the human ovarian cancer cell lines SK-OV-3, OvCAR-3 (ovarian carcinoma line established at Raven Biotechnologies); the human prostate cancer cell line PC3, or a mixture of the 3 cancer cell types. After incubation, the cell samples were washed, resuspended in 0.1 ml diluent and incubated with 1 μg/ml of FITC conjugated F(ab′)₂ fragment of goat anti-mouse IgG for 30 min at 4° C. The cells were washed, resuspended in 0.5 ml FACS diluent and analyzed using a FACScan cell sorter (Becton Dickinson; San Jose, Calif.). Hybridoma clones were selected for further expansion, cloning, and characterization based on their binding to the surface of one or more of the cell lines as assessed by FACS. A hybridoma making a monoclonal antibody designated mhoe-4 which binds an antigen designated Ag-hoe4 and an epitope on that antigen designated Ag-hoe4.1 was selected.

For screening a panel of antibodies against the antigen source, HOE cells were detached from tissue culture flasks in the presence of 0.5 mM EDTA, centrifuged at 1400 rpm for 5 minutes and resuspended in PBS containing 1% BSA and 2 mM EDTA (FACS diluent). The cells were counted and adjusted to 10⁷ cells/ml. About 0.1 ml of cells were incubated with 100 μl hybridoma supernatant or 1 μg of purified monoclonal antibodies in 100 μl FACS diluent for 30 min at 37° C. A hybridoma making a monoclonal antibody designated mhoe-4 which binds an antigen designated Ag-hoe4 and an epitope on that antigen designated Ag-hoe4.1 was selected

### Example 3

### Purification of mhoe-4

Monoclonal antibodies were purified from tissue culture supernatant using protein-G affinity chromatography. The following materials were used for the antibody purification process: hybridoma tissue culture supernatant, Immunopure (G) IgG binding buffer (Pierce #21011 Rockford, Ill.), Immunopure IgG Elution Buffer (Pierce #21009), concentrated HCl (for adjusting pH), Corning 1 liter PES (polyether sulfone), 0.22 μm filter (Coming #431098, Corning, N.Y.), Amersham Pharmacia GradiFrac System (Amersham Pharmacia, Piscataway, N.J.), Protein-G Sepharose 4 Fast Flow (Amersham Pharmacia #17-0618-02), Stripping buffer which is 3M KSCN/50 mM Tris pH 7.8, and PBS (phosphate buffered saline) 3M Tris pH 9.0.

To purify the mhoe-4 antibody, the volume of supernatant was measured and an equal volume of binding buffer was added to the supernatant. The mixture was allowed to equilibrate to room temperature. The supernatant was clarified by passage through a 0.22 μm filter. The supernatant was loaded on to a protein-G column using the GradiFrac system. The column was washed with 5-10 column volumes of binding buffer. The monoclonal antibodies were eluted with the elution buffer and 2 ml fractions were collected. An OD₂₈₀ reading of the fractions were obtained and the fractions containing monoclonal antibodies were pooled. The eluted monoclonal antibody fractions were neutralized by adding 1/20 volume of 3M Tris. The sample was dialyzed in 1×PBS at 4° C. (with 3 buffer changes of at least 3 hours per change). The purified monoclonal antibodies were sterile filtered (0.2 uM) and stored at 2-8° C.

After purification of the mhoe-4 monoclonal antibody from the hybridoma supernatant, it was re-tested for binding to HOE cells. The cell samples were prepared as described above in Example 7 and incubated with the purified antibody at various concentrations After incubation the cells were washed, resuspended in 0.1 ml diluent and incubated with 1 μg of FITC conjugated F(ab′)₂ fragment of goat anti-mouse IgG for 30 min at 4° C. The cells were washed, resuspended in 0.5 ml FACS diluent and analyzed using a FACScan cell sorter (Becton Dickinson; San Jose, Calif.). A shift to the right on the FACScan histogram indicated that the purified antibody still bound to the HOE cells.

### Example 4

### Identification and Characterization of Antigen Ag-hoe4

A cell pellet (approximately 25 ul packed cell volume of the pancreatic tumor cell line Rav9926 (established at Raven Biotechnologies, Inc.) was lysed by first diluting the cells to 0.5 ml in water followed by freezing and thawing three times. The solution was centrifuged at 14,000 rpm. The resulting pellet, containing the cell membrane fragments, was resuspended in 50 ul of SDS sample buffer (Invitrogen, Carlsbad, Calif.). The sample was heated at 80° C. for 5 minutes and then centrifuged for 2 minutes at 14,000 rpm to remove any insoluble materials. Other cell lines that express Ag-hoe4 and can be used for purification include SK-OV-3, LNnCaP, or the other cell lines which bind mhoe-4 as indicated in Table 3.

The samples were analyzed by Western blot using a 4 to 20% Tris-Glycine SDS polyacrylamide gradient gel (Invitrogen; Carlsbad Calif.) following the manufacturers' directions. Ten microliters of membrane sample were applied to one lane on the polyacrylamide gel. A separate ten microliter sample was reduced first by the addition of 2 μL of dithiothreitol (100 mM) with heating at 80° C. for 2 minutes and then loaded into another lane. The pre-stained molecular weight markers SeeBlue Plus2 (Invitrogen; Carlsbad, Calif.) were used to assess molecular weight on the gel. The gel proteins were transferred to a nitrocellulose membrane using a transfer buffer of 14.4 g/l glycine, 3 g/l of Tris Base, 10% methanol, and 0.05% SDS. The membranes were blocked, probed with the antibody mhoe-4 (at a concentration of 0.5 ug/ml), and developed using the Invitrogen WesternBreeze Chromogenic Kit-AntiMouse according to the manufacturer's directions. In both the reduced and non-reduced samples of the pancreatic tumor cell membrane samples, a very prominent band was observed migrating at about the same molecular weight as the alcohol dehydrogenase prestained marker (51 kDa). Therefore, the mhoe-4 antibody recognizes an antigen on a protein in pancreatic tumor cells that, based upon Western blot analysis using this molecular weight marker system, has a molecular weight of approximately 51 kDa.

Two much fainter bands were also detected: one appeared in both the reduced and non-reduced lanes midway between the alcohol dehydrogenase (50 kDa) and the glutamate dehydrogenase marker (64 kDa) markers, and a second faint band appeared only in the non-reduced sample, migrating between the phosphorylase B marker (1148 kDa) and the myosin marker (250 kDa).

### Example 5

### Isolation of Antigen Ag-hoe4 for Mass Spectrometry

Purified antibody mhoe-4 was concentrated to approximately 1 mg/ml using a Centricon YM30 concentrator (Millipore Cat. No. 4208). Approximately 1 mg of mhoe-4 was covalently coupled to 0.35 gram of cyanogen bromide-activated Sepharose 4B resin (Amersham Pharmacia Biotech Cat. No. 17-0430-01) according to the manufacturer's instructions. Freshly grown Rav9926 cells (˜2×10⁹ cells) were harvested from spinner flasks. The cells were pelleted centrifugally, then were resuspended in a total of 15 mL deionized water (dH2O) containing 100 μl of Protease Inhibitor Cocktail (Sigma Cat. No. P8340).

The cell suspension was frozen at −80° C., then thawed. This process was repeated for five cycles in order to disrupt the cells. The cell membranes were collected by centrifugation at 14,000 rpm for 15 minutes at 4° C. in an Eppendorf microcentrifuge.

The cell membrane pellet was resuspended in 2 ml of Hank's Balanced Salt Solution (HBSS, GibcoBRL Cat. No. 14175-079) containing 2% Empigen BB detergent (Calbiochem Cat. No. 324690) and 50 μl Sigma Protease Inhibitor Cocktail, pH 7.0. The cell membrane preparation was then placed on a rotator overnight at 4° C.

The cell membrane preparation was diluted with HBSS to a final concentration of 1% Empigen BB. Insoluble cell debris was removed by centrifugation at 14,000 rpm for 15 minutes at 4° C. in an Eppendorf microcentrifuge. The supernatant containing the soluble membrane proteins was collected and stored at −80° C. until used in affinity purification.

The cell membrane extract was thawed, then mixed with the previously prepared mhoe-4-affinity gel and rotated for 2 hours at 4° C. After incubation, the affinity gel was washed extensively as follows: 5 times with serum- and additive-free HBSS+1.0% Empigen BB→3 times with serum- and additive-free HBSS+0.5% Empigen BB→3 times with serum- and additive-free HBSS+0.25% Empigen BB→2 times with serum- and additive-free HBSS+0.125% Empigen BB→2 times with serum- and additive-free HBSS alone→1 time with 0.5 M NaCl in dH₂O→1 time with PBS.

Each wash consisted of 5.0 mL, with the exception of the 0.5 M NaCl wash, which was 1.5 ml. The antigen was then eluted from the affinity gel with 1.5 ml of 2% acetic acid in dH₂O for 2 minutes. The 0.5 M NaCl wash and the acid-eluted antigen were retained, and the sample volumes of each were reduced to ˜100 μl using a SpeedVac (Savant Cat. No. ISS110) on medium heat for ˜2.5 hours.

The samples were then precipitated and extracted by the addition of 400 μL methanol and 100 μL chloroform. Samples were vortexed, then 300 μl dH₂O was added and mixed gently. The samples were centrifuged at 14,000 rpm for 4 minutes at room temperature in an Eppendorf microcentrifuge. The protein localizes at the interface of the liquid phases, so most of the top layer was discarded. 400 μl methanol was added to the remainder of the samples and mixed gently. The samples were again centrifuged at 14,000 rpm for 4 minutes at room temperature. The supernatant was discarded, and a SpeedVac was used to dry the samples completely.

The dried samples were reconstituted by the addition of 28 μl 1×LDS sample buffer (Invitrogen Cat. No. NP007) in preparation for electrophoresis. The samples were heated to 75° C. for 10 minutes, then were centrifuged in a microcentrifuge and vortexed to mix. 25 μl of each sample was loaded into a single lane on a pre-cast NuPAGE 4-12% gradient gel (Invitrogen Cat. No. NP0322) for subsequent antigen identification. Two microliters were loaded in another lane for Western blotting analysis. Appropriate molecular weight standards were also included on the gel, as were samples of the cell membrane protein extract before and after incubation with the affinity resin. Electrophoresis was performed according to the manufacturer's instructions. The gel was fixed in 50% methanol containing 10% acetic acid for 30 minutes, then was stained using a Colloidal Blue stain (Invitrogen Cat. No. LC6025) according to the manufacturer's instructions. A non-fixed portion of the gel was transferred onto a nitrocellulose sheet (Invitrogen Cat. No. LC2000) for Western blotting, again according to the manufacturer's instructions. The blot was then probed with mhoe-4 and developed using a Western Blotting Kit (Invitrogen Cat. No. WB7103) to confirm antigen recognition.

Stained protein bands from the NuPAGE gel were excised using clean scalpel blades and were placed in clean Eppendorf tubes. Excised bands were stored at −20° C. until used for protein identification by mass spectrometry.

### Example 6

### Characterization of the Antigen to which mhoe-4 Binds Using MALDI Mass Spectrometry

The antigen to which mhoe-4 binds was isolated as described in Examples 4 and 5 and subjected to MALDI mass spectroscopy. Eluates of the immunoaffinity column were separated by SDS-PAGE, and the bands were excised and extracted. The gel slice was tryptically digested “in gel” (Gharahdaghi, F., Weinberg, C. R., Meagher, D. A., Imai, B. S., and Mische, S. M. (1999) Electrophoresis 20, 601-605). Extracted peptides were analyzed by matrix-assisted laser desorption/ionization time-of-flight mass spectrometry (MALDI-Tof) on a Kratos, AXIMA CRF. Peptide masses were determined within 100 ppm and a time ion gate with a curved field reflectron was employed for peptide isolation and fragmentation via post-source-decay (PSD). Searches were conducted with the Protein Prospector Programs (Clauser K. R., Baker, P. R., and Burlingame A. L., Analytical Chemistry 71, 2871-(1999) MSFit and MSTag. The antigen was identified as cytokeratin 8 (CK8). By Western blotting, mhoe-4 was shown to bind to CK8 but not cytokeratin 18 (CK18).

### Example 7

### Epitope Mapping

A systematic screening of the binding of mhoe-4 to 536 overlapping peptides derived from the CK8, consisting of 12 residue peptides, was performed. Using Pepscan Systems (P.O. Box 2098, 8203 AB Lelystad, The Netherlands), mhoe-4 was found to bind preferentially to the following multiple peptides (out of 536 tested): FLEQQNKMLETK (SEQ ID NO: 1) with a peak of 3175 (highest peak), QEKEQIKTLNNK (SEQ ID NO:2) with a peak of 1960, YQELMNVKLALD (SEQ ID NO:3) with a peak of 1090, NMQGLVEDFKNK (SEQ ID NO:4) with a peak of 899, PRAFSSRSYTSG (SEQ ID NO:5) with a peak of 925, SSAYGGLTSPGL (SEQ ID NO:6) with a peak of 908, EDIANRSRAEAE (SEQ ID NO:7) with a peak of 897. Among these seven peptides, mhoe-4 bound to FLEQQNKMLETK (SEQ ID NO: 1) with higher affinity than to other six peptides.

### Example 8

### Immunohistochemistry Methods

Frozen tissue samples were embedded in OCT compound and quick-frozen in isopentane with dry ice. Cryosections were cut with a Leica 3050 CM mictrotome at thickness of 5 μm and thaw-mounted on vectabound-coated slides. The sections were fixed with ethanol at −20° C. and allowed to air dry overnight at room temperature. The fixed sections were stored at −80° C. until use. For immunohistochemistry, the tissue sections were retrieved and first incubated in blocking buffer (PBS, 5% normal goat serum, 0.1% Tween 20) for 30 minutes at room temperature, and then incubated with the mhoe-4 and control monoclonal antibodies diluted in blocking buffer (1 μg/ml) for 120 minutes. The sections were then washed three times with the blocking buffer. The bound monoclonal antibodies were detected with a goat anti-mouse IgG+IgM (H+L) F(ab′)₂-peroxidase conjugates and the peroxidase substrate diaminobenzidine (1 mg/ml, Sigma cat. No. D 5637) in 0.1 M sodium acetate buffer pH 5.05 and 0.003% hydrogen peroxide (Sigma cat. No. H1009). The stained slides were counter-stained with hematoxylin and examined under Nikon microscope.

Table 1 shows a panel of ovarian cancers, cysts and normal tissues (22 samples) stained with mhoe-4. These samples were frozen in OCT and sectioned immediately before use as described above. The sections were incubated with mhoe-4 1 ug/ml) and secondary antibody as described above. Slides were scored for the presence (+) (“+”does not indicate relative intensity of the staining between samples) or absence (−) of mhoe-4 signal.

TABLE 1
Binding of mhoe-4 to various ovarian cancers, cysts, and tissues

| Item # | Pathology | Differentiation | Binding Result |
|---|---|---|---|
| 1 | Serous-adenocarcinoma | N/A | N/A |
| 2 | Serous-adenocarcinoma | 0 intermediate | + |
| 3 | Serous-adenocarcinoma | 1 intermediate | + |
| 4 | Serous-adenocarcinoma | 0 low | + |
| 5 | Serous-adenocarcinoma | 1 intermediate | + |
| 6 | Serous-adenocarcinoma | 1 high | + |
| 7 | Serous-adenocarcinoma | 0 low | + |
| 8 | Serous-adenocarcinoma | 0 low | − |
| 9 | Serous-adenocarcinoma | 0 low | + |
| 10 | Serous-adenocarcinoma | 0 low | + |
| | Total Sero-cancer | | Binding Ratio = |
| | Samples = 10 | | 89% |
| 11 | Metastatic ovarian Serous- | metastasis | + |
| | adenocarcinoma | | |
| 12 | Metastatic ovarian cancer | metastasis | + |
| 13 | Metastatic ovarian cancer | metastasis | + |
| 14 | Metastatic ovarian cancer | metastasis | + |
| | Total metastatic ovarian | | Binding Ratio = |
| | cancer samples = 4 | | 100% |
| 15 | Ovarian endometrioid | − | − |
| | carcinoma | | |
| 16 | Ovarian endometrioid | 0 low | − |
| | cancer | | |
| | Total ovarian endometrioid | | Binding Ratio = |
| | samples = 2 | | 0% |
| 17 | Ovarian cancer | − | + |
| 18 | Malignant Brenner's tumor | − | + |
| 19 | Ovarian cancer | 0 intermediate | + |
| 20 | Ovarian carcinoma | − | + |
| 21 | Mucino-cancer | intermediate | + |
| 22 | Ovarian teratocarcinoma | − | − |
| | Total Other Ovarian Cancer | | Binding Ratio = |
| | Samples = 6 | | 83% |
| | Total ovarian cancer | | Binding Ratio = |
| | samples = 22 | | 81% |
| 23 | Ovarian cysts | benign, non- | + |
| | | cancerous | |
| 24 | Ovarian cysts | benign, non- | − |
| | | cancerous | |
| 25 | Mucinous ovarian cysts | benign, non- | − |
| | | cancerous | |
| | Total cyst Samples = 3 | | Binding Ratio = |
| | | | 33% |
| 26 | Normal ovary | Normal adult | Surface |
| | | | epithelial + |

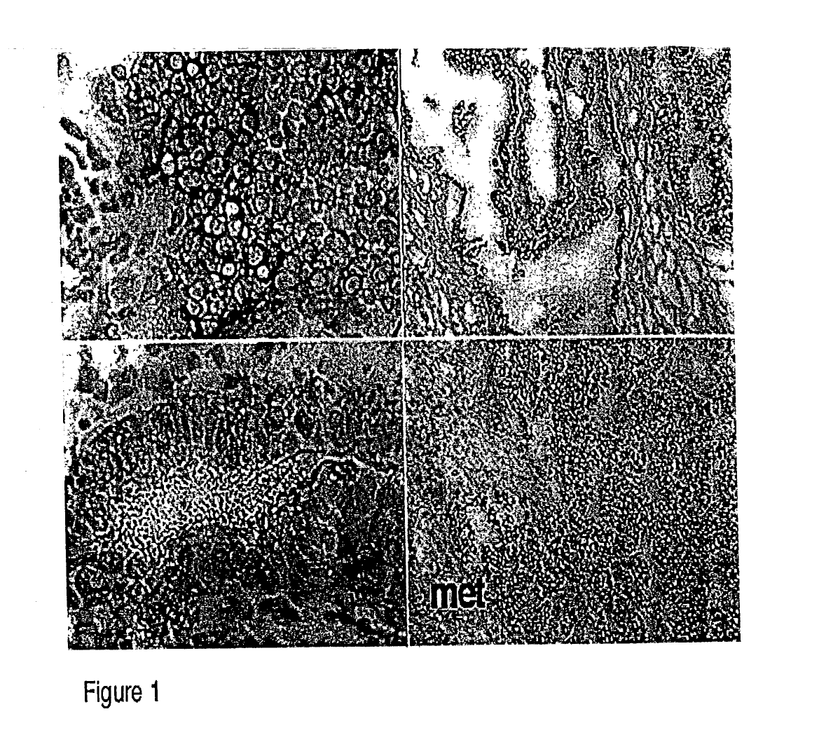

FIG. 1 shows results from three different patients' ovarian carcinoma tissues from Table 1 stained with mhoe-4. The fourth panel (lower right) is the staining of a metastatic tumor originating from another ovarian cancer. All cancer cells in the tissues were positive for the cell surface antigen binding mhoe-4.

In some cases, fixed paraffin embedded tissues were used for immunohistochemistry after appropriate antigen retrieval methods were employed. One such antigen retrieval method is described in Mangham and Isaacson, Histopathology 35:129-33 (1999). Other methods of antigen retrieval and/or detection may be used by one skilled in the art. Results from similar experiments performed using frozen tissues or, where appropriate, fixed tissue with antigen retrieval and polyMICA detection were performed. The binding of mhoe-4 to a variety of normal and cancer tissues was assessed. In all cases, antibody binding in control fixed tissues was correlated with that of frozen tissues. The results from frozen tissues were only used if the two did not match in the controls. For convenience, Table 2 shows the combined results of the staining of 7 major types of tumors with mhoe-4 using either fixed or frozen tumor tissues from 5 different sources, including those shown in Table 1 above. For each tumor type, the numbers of tumors testing positive for the Ag-hoe4.1 and the total number of such tumors tested is shown (+/total). The percentage of tumors binding mhoe-4 is also indicated.

TABLE 2
Summary of the incidence of the Ag-hoe4.1 antigen
occurrence on major tumor types

| Cancer | Ag-hoe4.1 | |
|---|---|---|
| Ovarian | 86.9% | (20/23) |
| Prostate | 100% | (11/11) |
| Lung | 85.7% | (6/7) |
| Colon | 88.8% | (8/9) |
| Pancreas | 100% | (1/1) |
| Breast | 87.5% | (7/8) |
| Renal | 100% | (6/6) |
| Thyroid | 100% | (2/2) |
| Total | 91% | (61/67) |

### Example 9

### Immunocytochemistry Result from CellArray™

Monoclonal antibody mhoe-4 was used to test reactivity with various cell lines from different types of tissues. Cells from different established cell lines were removed from the growth surface without using proteases, packed and embedded in OCT compound. The cells were frozen and sectioned, then stained using a standard IHC protocol. The CellArray™ technology is described in WO 01/43869. Results from the CellArray binding experiments are summarized in Table 3.

TABLE 3
Binding of the mhoe-4 antibody to established
human tumor and normal cell lines

| | | | Reactivity with |
|---|---|---|---|
| | | | mhoe-4 |
| Cell Lines | Organ | Type | (respectively) |
| SK-OV-3, CaOv3, | human ovary | cancerous | +, +, +, + |
| OvCAR-3, ES-2 | | | |
| PC3, LNCaP | human prostate | cancerous | +, + |
| HT29, SW480, | human colon | cancerous | +, +, + |
| Colo-205 | | | |
| A549, SKMES-1, | human lung | cancerous | +, +, + |
| Rav CA130 | | | |
| PANC-1, Capan-1, | human | cancerous | +, +, +, +, +, |
| HF700T, CFPAC-1, | pancreas | | +, + |
| HF-PAC-1, AsPC-1, | | | |
| Rav9926 | | | |
| SKBR3 | human breast | cancerous | + |
| hPED | human fetal | normal | + |
| | pancreas | | |
| HMEK, NHEK | human adult | normal | −, − |
| | endothelial | | |
| | cells | | |
| COS | monkey kidney | virus transformed | + |
| AoSMC | human aortic | normal | − |
| | smooth muscle | | |
| WI-38 | human fetal | normal | − |
| | lung fibroblasts | | |
| RL-65 | neonatal rat | normal | − |
| | lung | | |

### Example 10

### Binding of mhoe-4 to Normal Tissues

Normal tissue obtained by surgical resection were frozen and mounted as with tumor tissues. Cryosections were cut with a Leica 3050 CM mictrotome at thickness of 5 μm and thaw-mounted on vectabound-coated slides. The sections were fixed with ethanol at −20° C. and allowed to air dry overnight at room temperature. PolyMICA™ Detection kit was used to determine binding of mhoe-4 to normal tissue. Primary antibody mhoe-4 was used at a dilution of 1 to 100 (final concentration of 1 ug/ml). The results of staining of normal tissues with mhoe-4 is shown is Table 4.

TABLE 4
Staining of normal tissues using polyMICA

| Tissue | | mhoe-4 |
|---|---|---|
| Spleen | Vessel wall (smooth muscle) | − |
| | Lymphoid cells | − |
| | Endothelial cells | − |
| Fibrofatty tissue | Vessel wall (smooth muscle) | − |
| | Fibroblasts | − |
| | Endothelium | − |
| Skin | Vessel wall (smooth muscle) | − |
| | Epidermis | N/A |
| | Pilo-errector (Smooth muscle) | − |
| | Sweat glands | ++ |
| | Endothelium | − |
| | Fibroblasts | − |
| | Hair follicle | − |
| Tendon | Vessel wall (smooth muscle) | − |
| | Fibroblasts | − |
| | Endothelium | − |
| Nerve | Vessel wall (smooth muscle) | − |
| | Epineureum | − |
| | Schwann cells | − |
| | Fat | − |
| | Endothelium | − |
| | Fibroblasts | − |
| Skeletal muscle | Vessel wall (smooth muscle) | − |
| | Endothelium | − |
| Artery | Vessel wall (smooth muscle) | − |
| | Endothelium | − |
| Tonsil | Vessel wall (smooth muscle) | − |
| | Squamous epithelium | ++ (basal layer) |
| | Lymphocytes | − |
| | Endothelial cells | − |

Further screening of normal tissues was done, using tissues obtained by surgical resection and/or at autopsy, on both frozen and paraffin-embedded tissues. No staining was seen on normal adult human tissue in peripheral nerves, brain, skeletal muscle, heart, blood vessels, B-cells, T-cells, bone, adipocytes, spleen, adrenal gland, or skin epidermis, dermis, hair follicles, smooth muscle, or sebaceous glands. While the majority of the cells in the ovary and tonsil, did not stain, the epithelium in the capsule of these tissues was positive for mhoe-4 staining. In addition staining of a subset of the exocrine epithelium of the skin (sweat gland), prostate, lung, colon, duodenum, pancreas, breast, stomach, liver and uterus stained at some level at or below that seen on tumors. Staining of a subset of cells in the kidney was seen on some tissue samples (autopsy) but not others (surgical samples) and correlated negatively with the state of preservation of the tissue.

### Example 11

### Effect of mhoe-4 on the Proliferation of Human Ovarian Carcinoma Cells

Anchorage-independent growth was assessed by the ability of tumor cells to form colonies when suspended in an agar solution. SK-OV-3 cells are human ovarian carcinoma cell line obtained from ATCC (ATCC #HTB 77). SK-OV-3 clone 4 cells were derived by subcutaneously injecting SK-OV-3 cells into nu/nu mice allowing tumors to form, then removing and isolating cells from the tumors formed in nude mice and cultured again in medium (1 animal/culture passage).

To prepare soft agar for culture, the stock agar was heated in microwave oven until the gel melted. The agar solution was transferred to 40° C. water bath to cool in a bio-safety hood. When the temperature was equilibrated, 1/10 volume of 10×CMRL 1040 medium (warmed at 37° C.) was added to the agar solution and mixed. The solution was then diluted with warm F12/DMEM to make 0.5% agar.

To each well of a 12 well plate, 0.5 ml 0.5% agar was added and allowed to gel at room temperature to form a bottom layer. Then, SK-OV-3 cells were dissociated with trypsin/EDTA solution and counted with a hemocytometer. The cells were diluted in warm F12/DMEM and mixed with remaining 0.5% agar in a tube to make 0.3% agar and approximately 340 cells/ml. The mixture was then added to the top of 0.5% agar gel prepared above and allowed to gel at room temperature. After the agar solidified to a gel-like consistency, 100 μl of fetal bovine serum (for final v/v of 10%) and 100 μl of mhoe-4 (73 μg/ml) or PBS were added to the top of the gel. The cultures were then placed in a CO₂ incubator 5% CO₂ at 37° C. and permitted to grow for 21 days.

On day 21, the cultures were examined under microscope and viable cell colonies were counted under a stereo microscope. mhoe-4 monoclonal antibody inhibited ovarian carcinoma colony formation by 55%. A summary of the results is shown in Table 5.

### Example 12

### Effect of mhoe-4 on Cell Growth of Prostate Carcinoma Cells

To determine the effect of mhoe-4 on the growth of prostate carcinoma cells, agar solution, which evaluates anchorage-independent growth, was used to grow prostate carcinoma cells incubated with and without mhoe-4 antibody. LNCaP cells, which are a human prostate carcinoma derived-cell line (ATCC #CRL-1740) were used. The bottom agar layers were 0.5% agar Bacto-Agar (Difco Laboratories, Detroit, Mich.) diluted in DMEM+10% FBS. The cell suspensions in medium containing 0.3% agar in medium were added slowly to the solid bottom layer of agar to a final cell density of 2×10⁴ cells in a 60 mm dish. mhoe-4 was added at 10 ug/ml in top gel and the plates were allowed to gel at room temperature and subsequently incubated for 30 days at 37° C. in a humidified 5% CO₂ atmosphere. The cells were stained using 1 mL of MTT added at a final concentration of 0.23 mg/mL and colonies of approximately 6 cells or more were counted by visualization.

As shown in Table 5, mhoe-4 inhibited the growth of the LNCaP cells derived from prostate carcinoma cell growth by 90.6%.

TABLE 5
Inhibition of ovarian and prostate carcinoma cell growth by mhoe-4

| | | SK-OV- | | |
|---|---|---|---|---|
| | SK-OV-3 | 3 cells + | LNCaP cells | LNCaP cells + |
| Plate # | cells alone | mhoe-4 | alone | mhoe-4 |
| 1 | 27 | 7 | 67 | 1 |
| 2 | 27 | 18 | 12 | 9 |
| 3 | 28 | 12 | 26 | 0 |
| Average | 27.3 | 12.3 | 35 | 3.3 |
| % Inhibition | — | 55% | — | 90.6% |

### Example 13

### Antibody Activity Against Human Tumor Xenografts in vivo

Human ovarian tumor cells, SK-OV-3 (ATCC #HTB 77) were embedded in a collagen gel at 10⁵ cells/graft, (in subsequent experiments the inoculum was 5×10⁵/graft) and grafted underneath the kidney capsule of nude mice. The grafts were allowed to grow for seven days. On day seven, the first dose of mhoe-4 (100 ug/gram body weight of host) was administered I.P. in the treatment group. This was followed by three subsequent doses (50 ug/gram body of host) injected I.P. every three days. Control group received only PBS (0.2 ml).

Three days after the last injection the hosts were euthanized and grafts were harvested and fixed in 10% neutral formalin. Specimens were further analyzed by hematoxylin and eosin stain for morphology as well as Ki67 antibody (a rabbit anti-human Ki67 antigen antibody from Dako Corp., 6392 Via Real, Carpinteria, Calif. 93013) stain for cell proliferation following the manufacturer's instructions for staining formalin fixed tissues.

For immunohistochemistry staining for Ki67, tissues were harvested, fixed in 10% neutral formalin, dehydrated, and embedded in paraffin. Five micron sections were deparaffinized, rehydrated, and microwaved in antigen retrieval solution (Dako Corporation, Carpinteria, Calif.) for 15 minutes at high power. After slides were cooled, sections were blocked with 3% H₂O₂ for 15 minutes, washed in PBS. A rabbit anti-human Ki67 antigen (Dako, A0047) diluted 1:100 in PBS was applied for-overnight at 4° C. The next day the sections were washed in PBS then incubated with biotinylated secondary goat anti-rabbit IgG antibody (Dako, EO432) and avidin-biotin complex (Vectastain ABC kit, Vector labs, CA, USA). Immunoactivity for Ki67 antigen was visualized utilizing 3,3′ diaminobenzidine tetrachloride (DAB; Sigma). Sections of Ki67 stained cells and the total cells in the grafts of mhoe-4 treated and untreated were counted. The results were summarized in Table 6.

TABLE 6
Percentage Ki67 positive cells in the mhoe-4 treated and untreated
SK-OV-3 grafts

| | mhoe-4 treated | | | untreated | | |
|---|---|---|---|---|---|---|
| | Ki67 | | % Ki67 | Ki67 | Total | % Ki67 |
| Section | Positive | Total Cell | Positive | Positive | Cell | Positive |
| # | Cell Count | Count | Cell | Cell Count | Count | Cell |
| 1 | 264 | 2387 | 11.1% | 278 | 1168 | 23.8% |
| 2 | 193 | 2203 | 8.8% | 341 | 1661 | 20.5% |
| 3 | 168 | 2574 | 6.5% | 236 | 1265 | 18.7% |
| 4 | 191 | 2433 | 7.9% | 384 | 1596 | 24.1% |
| 5 | 75 | 1998 | 3.8% | 294 | 1087 | 27.0% |
| 6 | 273 | 2759 | 9.9% | 507 | 1719 | 29.5% |
| 7 | 448 | 1798 | 24.9% | 489 | 949 | 51.5% |
| 8 | 300 | 2589 | 11.6% | 607 | 1642 | 37.0% |
| 9 | 349 | 2754 | 12.7% | 554 | 1693 | 32.7% |
| 10 | 241 | 1924 | 12.5% | | | |
| 11 | 210 | 1635 | 12.8% | | | |
| 12 | 238 | 1719 | 13.8% | | | |
| 13 | 217 | 1878 | 11.6% | | | |
| 14 | 373 | 1785 | 20.9% | | | |
| 15 | 214 | 1007 | 21.3% | | | |
| 16 | 232 | 1630 | 14.2% | | | |
| 17 | 275 | 1705 | 16.1% | | | |
| 18 | 348 | 1986 | 17.5% | | | |
| Total | 4609 | 36764 | | 3690 | 12780 | |
| Average | 256.1 | 2042.4 | 13.2% | 410 | 1420 | 29.4% |

As shown in Table 6, there was about 56% decrease in Ki67 staining in mhoe-4 treated SK-OV-3 grafts as compared to untreated SK-OV-3 grafts, indicating there was about 56% decrease in SK-OV-3 cell proliferation in mhoe-4 treated SK-OV-3 grafts as compared to untreated SK-OV-3 grafts.

A similar experiment is performed with human prostate tumor cells, LNCaP (ATCC #CRL-1740).

### Example 14

### Internalization of mhoe-4 and Toxin-Conjugated Anti-Mouse IgG

Mab-ZAP (Advanced Targeting Systems, San Diego, Calif.) is anti-mouse IgG conjugated to saporin, a toxin which inhibits protein synthesis. This toxin is impermeable to the cell membrane. If a monoclonal antibody is bound to a cell-surface antigen which is internalizable, the toxin-conjugate can bind to the bound monoclonal and be internalized, eventually killing the cell. Being dependent upon internalization for demonstration of toxic activity, the Mab-ZAP can serve to evaluate whether or not a given surface antigen will serve as a suitable target for any toxin that is dependent upon internalization to express cell toxic effects. As such, the Mab-ZAP serves as a model for such internalization-dependent toxins such as maytansinoids and calicheamicins.

For testing the internalization of mhoe-4 and saporin conjugated anti-mouse IgG by tumor cells and effect of killing the tumor cells after internalization of saporin, human prostate tumor cells, LNCaP (ATCC #CRL-1740) were removed from stock flasks with 10 mM EDTA and centrifuged. Cells were resuspended at 50,000/ml in appropriate medium and 100 μl plated per well in 96 well plates. Antibody mhoe-4 was added immediately to appropriate wells as a 10× concentrate, to make a final concentration of 10 ug/ml. After 15 minutes at room temperature Mab-ZAP (Cat. # IT-04, Advanced Targeting Systems, San Diego Calif.) was added to appropriate wells as 10×concentrate, to make final concentrations from 0.001 pM to 10⁴ pM. After 4-6 days growth, MTT was added (stock 5 mg/ml PBS, 1:10 dilution in well) for 4 hrs at 37° C. The medium was then removed from all wells and 100 μl/well DMSO was added. The plates was gently swirled to solublize the blue MTT precipitate and the plates were read in a plate reader at 540 nm.

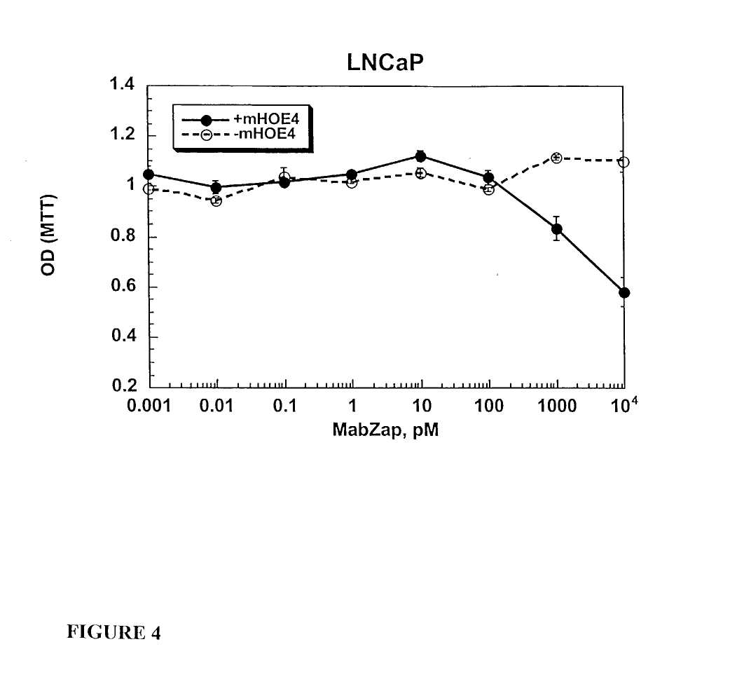

As shown in FIG. 4, there was a decrease in MTT staining in LNCaP in the presence of mhoe-4 as compared to the staining in the absence of mhoe-4 when Mab-ZAP was added above 100 pM, indicating the growth of human prostate tumor cells LNCaP was inhibited in the presence of mhoe-4 and Mab-ZAP and mhoe-4 and toxin-conjugated anti-mouse IgG was internalized in LNCaP. When Mab-ZAP was used at 10⁴ pM, there was about 50% of decrease in MTT staining, corresponding to about 50% inhibition of the growth of LNCaP by binding of mhoe-4 and Mab-ZAP.

However, there was no decrease in MTT staining in cancerous cell lines SK− OV-3 (human ovarian tumor cells, ATCC #HTB 77), SK-BR-3 (human breast tumor cells, ATCC #HTB 30), SK-MES-1 (human lung tumor cells, ATCC #HTB 58), or COLO 205 (human colon tumor cells, ATCC #CCL222) in the presence of mhoe-4 for four days as compared to the staining in the absence of mhoe-4 when Mab-ZAP was added between 0.001 pM to 10⁴ pM, indicating that Mab-ZAP was not internalized in these cells or was ineffective in growth inhibition in these cells under these experimental conditions.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

What is claimed is:

1. An antibody which preferentially binds to one or more peptides selected from the group consisting of FLEQQNKMLETK (SEQ ID NO: 1), QEKEQIKTLNNK (SEQ ID NO:2), YQELMNVKLALD (SEQ ID NO:3), NMQGLVEDFKNK (SEQ ID NO:4), PRAFSSRSYTSG (SEQ ID NO:5), SSAYGGLTSPGL (SEQ ID NO:6), and EDIANRSRAEAE (SEQ ID NO:7).

2. The antibody of claim 1, wherein the antibody preferentially binds to the peptide FLEQQNKMLETK (SEQ ID NO:1).

3. An antibody that preferentially binds to the same epitope on cytokeratin 8 as antibody mhoe-4 preferentially binds.

4. An antibody mhoe-4 produced by a host cell with a deposit number of ATCC No. PTA-3159 or progeny thereof.

5. A humanized antibody of the antibody mhoe-4 of claim 4.

6. The antibody of claim 1, wherein the antibody is linked to a therapeutic agent.

7. The antibody of claim 2, wherein the antibody is linked to a therapeutic agent.

8. The antibody of claim 3, wherein the antibody is linked to a therapeutic agent.

9. The antibody of claim 5, wherein the antibody is linked to a therapeutic agent.

10. A host cell line (ATCC No. PTA-3159) or progeny thereof.

11. A complex of cytokeratin 8 bound by antibody mhoe-4.

12. The complex of claim 11, wherein the antibody mhoe-4 is linked to a therapeutic agent.

13. A pharmaceutical composition comprising the antibody of claim 1 and a pharmaceutically acceptable excipient.

14. A pharmaceutical composition comprising the antibody of claim 2 and a pharmaceutically acceptable excipient.

15. A pharmaceutical composition comprising the antibody of claim 3 and a pharmaceutically acceptable excipient.

16. A pharmaceutical composition comprising the antibody of claim 5 and a pharmaceutically acceptable excipient.

17. A pharmaceutical composition comprising the antibody of claim 6 and a pharmaceutically acceptable excipient.

18. A pharmaceutical composition comprising the antibody of claim 7 and a pharmaceutically acceptable excipient.

19. A pharmaceutical composition comprising the antibody of claim 8 and a pharmaceutically acceptable excipient.

20. A pharmaceutical composition comprising the antibody of claim 9 and a pharmaceutically acceptable excipient.

21. A kit for detecting cancerous cells comprising an antibody according to claim 2 or 5.

22. A kit for inhibiting proliferation of cancerous cells comprising an antibody according to claim 2 or 5.

23. The kit of claim 22, wherein the antibody is linked to a therapeutic agent.

24. A method of generating antibody mhoe-4 comprising culturing a host cell (ATCC No. PTA-3159) or progeny under conditions that allow production of antibody mhoe-4, and purifying the antibody mhoe-4.

25. A method of detecting presence or absence of thyroid cancer cells in an individual comprising detecting cytokeratin 8 on thyroid cells from the individual.

26. The method of claim 25, wherein the cytokeratin 8 is detected by the antibody of any of claims 1-5.

27. A method of detecting presence or absence of cancerous cells in an individual comprising contacting cells from the individual with the antibody of claim 2 or 5, and detecting a complex of cytokeratin 8 and the antibody from the cells, if any.

28. The method of claim 27, wherein the cancerous cells that are detected are ovarian.

29. The method of claim 27, wherein the cancerous cells that are detected are prostate.

30. The method of claim 27, wherein the cancerous cells that are detected are lung.

31. The method of claim 27, wherein the cancerous cells that are detected are colon.

32. The method of claim 27, wherein the cancerous cells that are detected are pancreas.

33. The method of claim 27, wherein the cancerous cells that are detected are breast.

34. The method of claim 27, wherein the cancerous cells that are detected are renal.

35. A method of inhibiting proliferation of cancerous cells in an individual comprising administering a composition according to claim 14 or 16.

36. The method of claim 35, wherein the antibody is linked to a therapeutic agent.

37. The method of claim 35, wherein the cancerous cells are ovarian.

38. The method of claim 35, wherein the cancerous cells are prostate.

39. A method of delivering a therapeutic agent to cancerous cells in an individual comprising administering to the individual a composition according to claim 18 or 20.

40. A method of delivering a therapeutic agent into cancerous cells in an individual comprising administering to the individual a composition according to claim 18 or 20, wherein the cancerous cells are prostate.

41. The method of claim 40, wherein the therapeutic agent is saporin.
